(19) United States
(12) Patent Application Publication
(10) Pub. No.: US 2025/0129438 A1
(43) Pub. Date: Apr. 24, 2025

(54) ASSAY FOR DETECTION OF SARS-COV-2

(71) Applicant: Imam Abdulrahman Bin Faisal University, Dammam (SA)

(72) Inventors: Galyah ALHAMID, Dammam (SA); Huseyin TOMBULOGLU, Dammam (SA)

(73) Assignee: Imam Abdulrahman Bin Faisal University, Dammam (SA)

(21) Appl. No.: 18/489,441

(22) Filed: Oct. 18, 2023

(51) Int. Cl. C12Q 1/70

(52) U.S. Cl. CPC C12Q 1/701; C12Q 2600/16

(57) ABSTRACT

A method of detecting SARS-CoV includes amplifying isothermally a target sequence in a sample with at least one set of oligonucleotide primers and assaying the sample with a SARS assay to detect the target sequence of a SARS-CoV nucleic acid sequence. A kit for detecting SARS-CoV in a sample includes a reverse transcriptase, a universal primer set suitable for loop-mediated isothermal amplification (LAMP) of the target sequence in a SARS-CoV nucleic acid sequence and variants thereof containing mutations within one or more primer binding sites.

### STATEMENT OF PRIOR DISCLOSURE BY INVENTOR

Aspects of the present disclosure are described in G. Alhamid, H. Tombuloglu, D. Motabagani, D. Motabagani, A. A. Rabaan, K. Unver, G. Dorado, E. Al-Suhaimi, and T. Unver, “Colorimetric and fluorometric reverse transcription loop-mediated isothermal amplification (RT-LAMP) assay for diagnosis of SARS-CoV-2”; Functional & Integrative Genomics; 22; 1391-1401, incorporated herein by reference in its entirety. Aspects of the present disclosure are described in G. Alhamid, H. Tombuloglu, and E. Al-Suhaimi, “Development of Loop-mediated Isothermal Amplification (LAMP) Assays Using Five Primers Reduces the False-positive Rate in COVID-19 Diagnosis”; MedRxiv, incorporated herein by reference in it its entirety.

### STATEMENT OF ACKNOWLEDGEMENT

Support provided by Institute for Research and Medical Consultations (IRMC) under project number 2020-IRMC-S-3 and the Deanship of Scientific Research (DSR) of Imam Abdulrahman bin Faisal University (IAU) fund COVID19-2020-026-IRMC is gratefully acknowledged.

### REFERENCE TO SEQUENCE LISTING

In accordance with 37 CFR § 1.52(e)(5) and with 37 CFR § 1.831, the specification makes reference to a Sequence Listing submitted electronically as a .xml file named “546197-SEQL_syntheticconstruct_new”. The .xml file was generated on Oct. 17, 2023 and is 49,806 bytes in size. The entire contents of the Sequence Listing are hereby incorporated by reference.

### BACKGROUND

### Technical Field

The present disclosure is directed to detecting severe acute respiratory syndrome coronavirus (SARS-CoV) and particularly relates to a method and a kit for detecting SARS-CoV in a sample. The kit and method include amplifying a target sequence in the sample isothermally with at least one set of oligonucleotide primers and assaying the sample with a SARS assay to detect the target sequence of a SARS-CoV nucleic acid sequence.

### Description of Related Art

The “background” description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Coronavirus disease 2019, or COVID-19, is a highly contagious disease caused by a virus known as severe acute respiratory syndrome coronavirus 2 (SARS-CoV-2). Several virus variants have been identified and classified based on the severity of transmission of the COVID-19 disease. The disease was first reported in 2019 and soon led to a worldwide pandemic, causing millions of deaths and more than a hundred million detected and undetected infections. Prevention in containing the disease has been found more effective than a cure, especially in the absence of a single potent vaccine to curb variants of SARS-CoV-2. Structurally, the SARS-CoV-2 virus is a positive-sense single-stranded ribonucleic acid (RNA) virus with a genome consisting of 29,900 nucleotides enclosing five open reading frames (ORFs) (5′-3′). The ORFs include ORFlab polyprotein (P, 7,096 amino acids), spike glycoprotein (S, 1,273 amino acids), nucleocapsid protein (N, 419 amino acids), an envelope protein (E, 75 amino acids), and membrane protein (M, 222 amino acids) [Liu R, Fu A, Deng Z, Li Y, Liu T. Promising methods for detection of novel coronavirus SARS-CoV-2. View. 2020; 1(1):e4; and Khan S, Tombuloglu H, Hassanein S E, Rehman S, Bozkurt A, Cevik E, et. al. Coronavirus diseases 2019: current biological situation and potential therapeutic perspective. European Journal of Pharmacology. 2020; 886:173447. pmid:32763302, both of which are incorporated herein by reference in their entirety]. One of the major challenges that COVID-19 presents is early and accurate detection to contain the spread of the disease. The fact that the disease can be transmitted to others by asymptomatic individuals is another challenge. This necessitates early detection, fast diagnosis, and non-invasive and reliable methods to detect the SARS-CoV-2 virus.

Current approaches for SARS-CoV-2 diagnosis rely on quantitative reverse transcription polymerase chain reaction (RT-qPCR) tests. The RT-qPCR analysis detects the presence of viral nucleic acid in a sample with high sensitivity and specificity. The reliance on RT-qPCR tests is such that the World Health Organization (WHO) approved RT-qPCR as the standard test for SARS-CoV-2 detection. However, an RT-qPCR assay has shortcomings that, in a pandemic situation, translate into a loss of lives and increased infection spread. The RT-qPCR tests require complex equipment, extensive training, and multiple hours to complete the procedure. The rapid growth of a pandemic puts pressure on healthcare systems to cope with the rise in potential infections. Additionally, there are limited PCR laboratories due to the high resource requirements in establishing PCR laboratories. Further, due to the increased global demand for PCR consumables, their availability is limited to cater to the needs of the less privileged countries. As a result, the number of tests performed remains sub-optimal in many countries compared to the developed world.

Other solutions that are widely utilized include COVID-19 serology tests. The serological tests detect antibodies or antigens associated with the virus infection. Advantages of the serological tests include serological tests are easy to use and provide rapid results. In addition, the tests have minimal equipment and are cost-effective. However, serology immunoassays show a severe drawback of lack of the accuracy to be a reliable SARS-CoV-2 diagnostic test. In addition, the tests show low sensitivity and a high rate of false positive results.

One of the solutions for COVID-19 detection explored as an alternative for conventional PCR is loop-mediated isothermal amplification (LAMP). LAMP work is similar to the conventional PCR test with advantages. One advantage of LAMP over conventional PCR is that nucleic acid amplification occurs at a single temperature. Specific equipment, such as a thermal cycle, is mandatory for performing PCR. LAMP testing works independently of such equipment. Another advantage of LAMP over conventional PCR is a shorter reaction time. Thus, the LAMP assays are quicker, easier to use, and more cost-effective than RT-qPCR assays for diagnosis. One more advantage of the LAMP method is the ability to work in wide pH and temperature ranges. Yet another advantage of the LAMP assay is the ability of the assay to accept minimally processed samples. The LAMP assays also support flexible readout methods and can be confirmed via the naked eye. The LAMP assays specificity and sensitivity are as much as provided by the PCR tests. With the advantages the LAMP method has, some disadvantages and problems with rapid testing, and thus controlling the transmission of infection, still exist.

Another challenge that has emerged since the pandemic started is the rate of mutations that a SARS-CoV-2 genome can experience including: alpha, beta, gamma, delta, delta plus, epsilon, zeta, eta, theta, iota, kappa, lambda, and other known or unknown mutated forms of the virus. Variants and mutations of the virus, known and unknown, will continue to evolve and adapt, and vaccinations may not protect against them. Detecting and diagnostic tests may be an alternative to a vaccine.

Thus, diagnosis remains a factor in preventing the spread of COVID-19 and preventing infections. Furthermore, rapid, low-cost, and user-friendly methods help to address infectious disease outbreaks. To contain pandemics such as COVID-19, shortcomings associated with current methods, including LAMP, must be addressed. LAMP is an innovative gene amplification technique and an assay of a LAMP product shows promise as a detection tool; however, dealing with problems such as mismatch and misamplification can prove beneficial in controlling the pandemic, and thus end the global spread of infections. In addition, early identification and isolation of infected individuals helps the success of COVID-19 control. Therefore, there is a need for an accurate, simple, cost-effective, portable, scalable, and broadly applicable method and system to deal with a pandemic such as COVID-19.

Although numerous approaches have been developed since the pandemic started, there still exists a need to develop an accurate, user-friendly robust, and reliable assay that overcomes the limitations of the current art.

Accordingly, an object of the present disclosure is to provide a composition and methods of primers and assays for the detection of the SARS-CoV-2 RNA virus using loop-mediated isothermal amplification.

### SUMMARY

In an exemplary embodiment, a method of detecting SARS-CoV in a sample is disclosed. The method includes contacting the sample with a primer set and one or more reverse transcription polymerase chain reaction (RT-PCR) reagents to form a reaction mixture, wherein the primer set is selected from the group consisting of N-ID5 (Set-1), N-ID15 (Set-2), N-ID15n1L (Set-3), S-ID17 (Set-4), S-ID24 (Set-5), E-ID1 (Set-7), and RdRp-ID37 (Set-8). The method includes reverse transcribing a target sequence of a SARS-CoV nucleic acid sequence in the sample into complementary DNA (cDNA). The method includes amplifying the cDNA by incubating the reaction mixture at a temperature and for a time sufficient to amplify the target sequence of the SARS-CoV nucleic acid sequence in the sample. The method includes assaying the sample with a SARS assay to detect the amplified target sequence of the SARS-CoV nucleic acid sequence. The method includes detecting a presence of the amplified target sequence of the SARS-CoV nucleic acid sequence, wherein the presence of the amplified target sequence is indicative of SARS-CoV in the sample.

In some aspects of the present disclosure, a method of detecting SARS-CoV in a sample is disclosed. The method includes contacting the sample with a primer set and one or more reverse transcription polymerase chain reaction (RT-PCR) reagents to form a reaction mixture, wherein the primer set is S-set11 (Set-6). The method includes reverse transcribing a target sequence of a SARS-CoV nucleic acid sequence in the sample into complementary DNA (cDNA). The method includes amplifying the cDNA by incubating the reaction mixture at a temperature and for a time sufficient to amplify the target sequence of the SARS-CoV nucleic acid sequence in the sample. The method includes assaying the sample with a SARS assay to detect the amplified target sequence of the SARS-CoV nucleic acid sequence. The method includes detecting a presence of the amplified target sequence of the SARS-CoV nucleic acid sequence, wherein the presence of the amplified target sequence is indicative of SARS-CoV in the sample.

In some embodiments, the primer set is E-ID1 and includes five primers. The primers have at least sequences of forward outer (F3), forward inner (FIP), backward outer (B3), backward inner (BIP), and loop backward (LB).

In some embodiments, the primer set includes N-ID5, N-ID15, N-ID15n1L, S-ID17, S-ID24, and RdRp-ID37 and includes six primers. The primers have at least sequences of forward outer (F3), forward inner (FIP), loop forward (LF), backward outer (B3), backward inner (BIP), and loop backward (LB).

In some embodiments, the RT-PCR reagents include at least one reverse transcriptase, at least one deoxyribose nucleic acid (DNA) polymerase, deoxyribonucleotide triphosphates (dNTPs), magnesium ions, betaine or bovine serum albumin (BSA), buffer, and optionally colorimetric and fluorometric dyes.

In some embodiments, the amplifying is reverse-transcription loop-mediated isothermal amplification (RT-LAMP).

In some embodiments, the method further includes extracting nucleic acid from the sample.

In some embodiments, the SARS assay is a colorimetric RT-LAMP assay and a fluorometric RT-LAMP assay.

In some embodiments, the SARS assay is the colorimetric RT-LAMP assay. The assaying includes mixing a master mix, a primer mix, the sample, and dH₂O to form a colorimetric RT-LAMP mixture. The method includes incubating the colorimetric RT-LAMP mixture in a water bath at a constant temperature for at least 30 minutes.

In some examples of the above embodiment, the SARS assay is the fluorometric RT-LAMP assay. The assaying includes mixing a ribonucleic acid (RNA) buffer, an aqueous salt solution, a dNTP solution, a fluorescent dye, an RNA enzyme, a primer mix, the sample, and dH₂O to form a fluorometric RT-LAMP mixture. The method includes reacting the fluorometric RT-LAMP mixture in a PCR system at a constant temperature for a set number of cycles.

In some examples of the above embodiments, the SARS assay further includes adding guanidine hydrochloride (GuHCl) to the colorimetric RT-LAMP assay and the fluorometric RT-LAMP assay.

In some embodiments, the SARS-CoV is selected from the group including SARS-CoV-2, SARS-CoV-HKU-39849, SARS-CoV-TW11, SARS-CoV-TW4, SARS-CoV-Sin846, SARS-CoV-GZ0402, and SARS-CoV-Tor2.

In some embodiments, the SARS-CoV is SARS-CoV-2, and the SARS-CoV-2 includes at least one variant selected from the following group including alpha (B.1.1.7, Q.1-Q.8), beta (B.1.351, B.1.351.2, B.1.351.3), gamma (P.1, P.1.1, P.1.2), delta (B.1.617.2), and Omicron (B.1.1.529).

In some embodiments, the target sequence of a SARS-CoV nucleic acid sequence is located within at least one gene selected from the group including nucleocapsid (N), spike(S), RNA-dependent RNA polymerase (RdRp), and envelope (E) of a SARS-CoV genome.

In some embodiments, the detection of the presence of the amplification product for the target sequence of a SARS-CoV nucleic acid sequence includes an accuracy of at least 94% for the colorimetric RT-LAMP assay and the fluorometric RT-LAMP assay. The accuracy is based on colorimetric RT-LAMP assay results and fluorometric RT-LAMP assay results compared to reverse transcription real-time polymerase chain reaction (RT-qPCR) assay results.

In some embodiments, the primer set includes E-ID1 and has a limit of detection of 20 copies/μL.

In some embodiments, the primer set includes E-ID1 and amplifies a conserved region in the SARS-CoV-2 E gene as early as 20 minutes with no cross-reactivity with other respiratory viruses, including other SARS. The SARS assay has a specificity of at least 96% for the colorimetric RT-LAMP assay and the fluorometric RT-LAMP assay. The specificity is based on colorimetric RT-LAMP assay results and fluorometric RT-LAMP assay results compared to RT-qPCR assay results.

In some embodiments, the primer set includes E-ID1 and the target sequence in the sample was amplified within 20 minutes and up to 120 minutes. The SARS assay has a sensitivity of at least 89% for the colorimetric RT-LAMP assay and the fluorometric RT-LAMP assay. The sensitivity is based on colorimetric RT-LAMP assay results and fluorometric RT-LAMP assay results compared to RT-qPCR assay results.

In some embodiments, the method further comprises treating a subject for which the sample was obtained.

In some aspects of the present disclosure, a kit for use in detection of SARS-CoV in a sample is disclosed. The kit includes a reverse transcriptase, a universal primer set suitable for loop-mediated isothermal amplification (LAMP) of the target sequence in a SARS-CoV nucleic acid sequence and variants thereof containing mutations within one or more primer binding sites. The universal primer set suitable for LAMP is capable of hybridizing to the target sequence of a SARS-CoV nucleic acid sequence in the presence of a plurality of undefined mutations and is configured to provide a positive result for the target sequence of a SARS-CoV nucleic acid sequence in a predetermined assay time period otherwise determined for a positive sample of a target nucleic acid having a known sequence. The kit further includes the reagents where the reagents may be combined in a mixture in a single container or provided in separate containers.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative embodiments and the following detailed description thereof are merely exemplary aspects of the teachings of this disclosure and are not restrictive.

### BRIEF DESCRIPTION OF DRAWINGS

A better understanding of embodiments of the present disclosure (including alternatives and/or variations thereof) may be readily obtained with reference to the following detailed description of the embodiments when considered in connection with the accompanying drawings, wherein:

FIG. 1A shows a colorimetric test using primer sets on positive controls (PC) and non-template controls (NTC), according to certain embodiments;

FIG. 1B shows fluorometric results using the primer sets showing a fluorescent amplification in PC, according to certain embodiments;

FIG. 2A shows a colorimetric reverse-transcription loop-mediated isothermal amplification (RT-LAMP) using an E-ID1 primer set on clinical samples before and after optimization, according to certain embodiments;

FIG. 2B shows a fluorometric RT-LAMP before optimization on different clinical samples, according to certain embodiments;

FIG. 2C shows the fluorometric RT-LAMP after optimization on different clinical samples, according to certain embodiments;

FIG. 3A depicts a limit of detection (LoD) of E gene primers tested in the colorimetric RT-LAMP assay on serially diluted severe acute respiratory syndrome coronavirus 2 (SARS-CoV-2) synthetic ribonucleic acid (RNA) control, according to certain embodiments;

FIG. 3B depicts validation of the colorimetric LoD results via loading an RT-LAMP product post-reaction in 2% agarose gel showing band patterns of the detected diluted samples, according to certain embodiments;

FIG. 4A shows an optimized colorimetric RT-LAMP assay tested on SARS-CoV-2 clinical samples, according to certain embodiments;

FIG. 4B shows an optimized fluorometric RT-LAMP assay tested on SARS-CoV-2 clinical samples, according to certain embodiments;

FIG. 4C shows loading eight samples in 2% agarose stained with a deoxyribonucleic (DNA) stain and a 0.05-10 Kb DNA ladder used as a marker, according to certain embodiments;

FIG. 5A is a plot depicting the quantification of the color change between positive and negative samples by the spectrophotometric measurement of optical density (ΔOD) at 434 nm and 560 nm, according to certain embodiments;

FIG. 5B depicts the time to detection threshold of positive samples, according to certain embodiments;

FIG. 5C depicts the percentage of total positive samples detected plotted against time, according to certain embodiments;

FIG. 6A depicts E-ID1 primers' binding sites on the E gene of SARS-CoV-2 variants of concern (VOC) and other SARS viruses (SEQ ID NO.: 48, SEQ ID NO.: 49, SEQ ID NO.: 50, SEQ ID NO.: 51, SEQ ID NO.: 52, SEQ ID NO.: 53, SEQ ID NO.: 54, and SEQ ID NO.: 55), wherein emboldened letters correspond to a mismatched nucleotide, according to certain embodiments; and

FIG. 6B depicts a selectivity of E gene primers calorimetrically tested against clinical samples infected with SARS-CoV-2 and other respiratory viruses (1-8), including coronavirus 229 E, parainfluenza virus 3, parainfluenza virus 4, human metapneumovirus A+B, bocavirus, enterovirus, and rhinovirus, according to certain embodiments.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. Wherever possible, corresponding, or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values there between.

The term “master mix” refers to a combination of reagents which can be added to a sample to execute a reaction in an assay where the combination enhances the efficiency and speed of performing the assay. The master mix described herein may include reagents to reduce non-specific amplification. The master mix may include a mesophilic strand displacing DNA polymerase and may additionally include other enzymes such as a reverse transcriptase, uracil deglycosylase (also referred to herein as uracil DNA glycosylase (UDG)) for example, a thermolabile UDG which becomes inactivated in the temperature range of 50 to 60° C. or at 65° C.; and a thermolabile Proteinase K such as a thermolabile Proteinase K. The master mix may also include a reversible inhibitor of DNA polymerase activity. An example of a reversible inhibitor is an oligonucleotide known as an aptamer that binds to the DNA polymerase and blocks its activity below a selected temperature (for example, 50, 55, or 60° C.) but above that temperature, the oligonucleotide is disassociated from the enzyme, permitting the reverse transcriptase to become active. In some embodiments, the master mix includes a reverse transcriptase and a reversible inhibitor of reverse transcriptase activity for inhibiting the activity of these enzymes below 40, 45, or 50° C. An example of a reversible inhibitor is an oligonucleotide known as an aptamer that binds to the reverse transcriptase and blocks activity below a certain temperature (for example, 40° C.) but above that temperature, the oligonucleotide is disassociated from the enzyme, permitting the reverse transcriptase to become active. This allows setting up a reaction at room temperature while avoiding non-specific amplification. The master mix may also comprise inhibitors of nucleases such as RNase inhibitors and/or DNase inhibitors. These inhibitors may be chemical reagents such as poloxamers, and/or aptamers. However, nucleases may also be inactivated by submitting the sample combined with a lysis buffer to a high temperature for an effective time. The master mix may also include dNTPs such as dTTP, dATP, dGTP, and dCTP as well as dUTP for carryover prevention. For example, a 2× master mix may contain the dNTPs in equal quantities except the dUTP at 50% concentration of the other dNTPs. The master mix may include single strand binding proteins and/or helicases to reduce non-specific amplification. The master mix may also include a dye that can be a pH-sensitive or metallochromic dye. The master mix may be lyophilized or freeze-dried. It may be preserved for storage in a suitable buffer that may contain at least one reducing agent and at least one buffer that may contain at least one reducing agent and at least one detergent capable of storage at −20° C. for an extended period (for example, months). The inclusion of a reducing agent may be desirable if RNA is the template nucleic acid. The master mix for use in pH colorimetric LAMP may have a low buffer concentration, such as 5 mM Tris or less. A low buffer concentration is not required if a metallochromic dye of fluorescent dye is used to detect amplification. The master mix may be prepared in a 2×, 3×, 4×, 5×, 10×, or any suitable concentration. The master mix, once diluted by the sample, will result in a 1× concentration. The master mix may contain primers, or primers are not contained in the master mix.

As used herein, the term “primer” refers to a short single-stranded oligonucleotide sequence complementary to a nucleic acid strand to be replicated. It may serve as a starting point for synthesizing a primer extension product. It may act as a point of initiation of synthesis when placed under conditions in which synthesis of primer extension product, which is complementary to a nucleic acid strand (template), is induced and/or in the presences of nucleotides and an agent for polymerization, such as DNA polymerase, and at a suitable temperature and pH.

As used herein, the term “oligonucleotide(s)” refers to short DNA and/or RNA molecules, oligomers or oligos, used in a range of genetic testing and research. Oligonucleotides may be synthesized in a laboratory by solid-phase chemical synthesis and may be single-stranded molecules with any user-specified nucleic acid sequence. Oligonucleotides may be used for artificial gene synthesis, polymerase chain reaction (PCR), DNA sequencing, molecular cloning, as molecular probes, and the like. Oligonucleotides bind in a sequence-specific manner, to their respective complementary oligonucleotides, DNA, or RNA to form duplexes or, less often, hybrids of a higher order. Examples of procedures that use oligonucleotides include, but are not limited to, DNA microarrays, Southern blots, fluorescent in situ hybridization (FISH), PCR, the synthesis of artificial genes, and any procedure known in the art.

As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases capable of hybridizing to at least one nucleic acid strand even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single stranded nucleic acid molecule.

Aspects of the present disclosure are directed to a method of detecting SARS-CoV in a sample, such as a clinical sample. Such detection may be accomplished in situ or in vitro but is preferably conducted in vitro. As used herein, “sample” refers to any material or mixture of materials containing one or more analytes or entities of interest. As used herein, “analyte” refers to a substance that is suitable for testing in the present disclosure. The clinical samples that may be evaluated in accordance with the present disclosure include any that may contain SARS-CoV, and include blood samples, bronchoalveolar lavage fluid specimens, fecal samples, fibrobronchoscope brush biopsy samples, nasal swab samples, nasopharyngeal swab samples, pharyngeal swab samples, oral samples (including saliva samples, sputum samples, etc.) and urine samples. Preferably, however, the employed clinical sample will be a nasal swab sample, a nasopharyngeal swab sample, a pharyngeal swab sample, or a sputum sample, and most preferably, the employed clinical sample will be a nasopharyngeal swab sample. In an embodiment, the sample may be pretreated to extract RNA that may be present in the sample. Alternatively, and more preferably, the sample will be evaluated without prior RNA extraction.

The method includes contacting the sample with a primer set and one or more reverse transcription polymerase chain reaction (RT-PCR) reagents to form a reaction mixture. In an embodiment, the RT-PCR reagents may comprise at least one reverse transcriptase, at least one deoxyribose nucleic acid (DNA) polymerase, deoxyribonucleotide triphosphates (dNTPs), magnesium ions, betaine or bovine serum albumin (BSA), a buffer, optionally colorimetric and fluorometric dyes, and any RT-PCR reagents known in the art. As used herein, the term “oligonucleotide primer(s)” denotes a nucleic acid molecule that comprises at least 10 nucleotide residues and not more than 500 nucleotide residues, more preferably, not more than 200 nucleotide residues, still more preferably, not more than 100 nucleotide residues, and still more preferably, not more than 50 nucleotide residues, and that is capable of specifically hybridizing to the target sequence. Typically, an oligonucleotide contains a 5′ phosphate at one terminus (“5′ terminus”) and a 3′ hydroxyl group at the other terminus (“3′ terminus”) of the chain. The most 5′ nucleotide of an oligonucleotide may be referred to herein as the “5′ terminal nucleotide” of the oligonucleotide. The most 3′ nucleotide of an oligonucleotide may be referred to herein as the “3′ terminal nucleotide” of the oligonucleotide. As used herein, the term “hybridize” refers to a process of formation of double stranded nucleic acid regions between one, two, or many single stranded nucleic acid molecules complementary to one, two, or many target nucleic acid sequences. Hybridization may occur through specific hydrogen bonds between standard (Watson-Crick) base pair(s). Nucleic acid(s) that are “complementary” or “complement(s)” are those that are capable of base-pairing according to the standard Watson-Crick, Hoogsteen, or reverse Hoogsteen binding complementarity rules. As used herein, the term “specifically hybridizing” denotes the capability of a nucleic acid molecule to detectably anneal to another nucleic acid molecule, preferably a complementary nucleic acid molecule, under conditions in which such nucleic acid molecule does not detectably anneal to a non-complementary nucleic acid molecule. As used herein, the term “nucleic acid” includes both deoxyribonucleic acid (DNA) and ribonucleic acid (RNA), including DNA and RNA containing non-standard nucleotides. A “nucleic acid” contains at least one polynucleotide (a “nucleic acid strand”). The term “nucleic acid”, as used herein, refers to either deoxyribonucleic acid (DNA), ribonucleic acid (RNA), single-stranded or double-stranded, and any chemical modifications thereof. The nucleic acid detected according to the systems, assays, and/or methods disclosed herein may be a full-length nucleic acid or a fragment thereof. Nucleic acids are biopolymers and macromolecules comprising nucleotides. Nucleotides are monomer components comprising a five-carbon sugar, a phosphate group, and a nitrogenous base. The nitrogenous base, or nucleobase, may be adenine “A”, cytosine “C”, guanine “G”, thymine “T”, uracil “U”, and any nitrogenous base known in the art. The primer set may comprise one or more primers. The primers may comprise one or more oligonucleotide primers. In an embodiment, the primer set is selected from the group consisting of N-ID5 (Set-1), N-ID15 (Set-2), N-ID15n1L (Set-3), S-ID17 (Set-4), S-ID24 (Set-5), E-ID1 (Set-7), and RdRp-ID37 (Set-8).

As used herein, a “polynucleotide” refers to a polymeric chain containing two or more nucleotides, which contain deoxyribonucleotides, ribonucleotides, and/or their analog, such as those containing modified backbones (e.g., peptide nucleic acids (PNAs) or phosphorothioates) or modified bases. Polynucleotides includes primers, oligonucleotides, nucleic acid strand, etc. A polynucleotide may contain standard or non-standard nucleotides. Thus, the term includes mRNA, tRNA, rRNA, ribozymes, DNA, cDNA, recombinant nucleic acids, branched nucleic acids, plasmids, vectors, probes, primers, etc. A “nucleic acid” may be single-stranded or double-stranded. The term “nucleic acid” refers to nucleotides and nucleosides which make up, for example, DNA macromolecules and RNA macromolecules. The most common nucleic acids are DNA and RNA. In some embodiments, the target sequence is DNA or RNA.

The method includes reverse transcribing a target sequence of a SARS-CoV nucleic acid sequence in the sample into complementary DNA (cDNA). Reverse transcription may take an RNA sequence, for example the target sequence of the SARS-CoV nucleic acid sequence, and synthesis a double-stranded DNA product. This process may be done with the use of one or more RNA-dependent DNA polymerases, known as reverse transcriptases.

The method includes amplifying the cDNA by incubating the reaction mixture at a temperature and for a time sufficient to amplify the target sequence of the SARS-CoV nucleic acid sequence in the sample. As used herein, the term “amplifying” or “amplification” denotes a process by which the number of copies of a gene is increased without a proportional increase in other genes. In some embodiments, the temperature may be 40 to 90° C., preferably 50 to 80° C., more preferably 60 to 70° C., and yet more preferably about 65° C. In some embodiments, the time may be 5 to 120 minutes, preferably 10 to 100 minutes, preferably 15 to 80 minutes, preferably 20 to 60 minutes, more preferably 25 to 40 minutes, and yet more preferably about 30 minutes.

The method further comprises assaying the sample with a SARS assay to detect the amplified target sequence of a SARS-CoV nucleic acid sequence. In some embodiments, the amplification is carried out without mis-amplification or a non-specific amplification. As used herein, the term “mis-amplification” denotes the incorrect amplification of a gene.

The method further includes detecting a presence of the amplified target sequence of the SARS-CoV nucleic acid sequence, thereby detecting SARS-CoV in the sample. Detecting SARS-CoV in the sample may be any positive assay result. Detecting SARS-CoV in the sample may be a result of a change in color that may be visible to a human eye or with the aid of a spectrophotometer, a change in fluorescence, a combination thereof, and a change in any other signal known in the art.

In an embodiment, the method comprises contacting the sample with a primer set and the primer set is S-set11 (Set-6).

In some embodiments, the amplifying is loop-mediated isothermal amplification (LAMP) and, more specifically, reverse-transcription loop-mediated isothermal amplification (RT-LAMP). LAMP is a nucleic acid amplification method that relies on auto-cycle strand-displacement DNA synthesis performed by Bst DNA polymerase, or other strand displacement polymerases. In LAMP, four primers recognize six unique target sequences on the template strand. Two of the primers are designated as “inner primers” (FIP and BIP) and two are designated as “outer primers” (F3 and B3). The reaction is initiated by annealing and extension of a pair of loop-forming primers (FIP and BIP), followed by annealing and extension of a pair of flanking primers (F3 and B3). Extension of these primers results in strand-displacement of loop-forming elements, which fold up to form hairpin-loop structures. In addition to containing a sequence that is complementary to a target sequence at their 3′ ends, the inner primers also contain a tail that comprises a sequence that is downstream of the 3′ end of primers in the template. Thus, an extension of an inner primer results in a product that has a self-complementary sequence at the 5′ end. Displacement of this product by an outer primer generates a product that has a loop at the 5′ end. Thus, the primer sets used in LAMP typically contain four, five, or six template-complementary sequences, where four sequences are found at the 3′ ends of the primers and two of the sequences are found at the 5′ ends of the primers. The initial reaction in LAMP results in a DNA product that has a dumbbell-like structure. In this product, the ends form stem loops and the single stranded region in between the stem loops is copied from the template. This product self-primes its own amplification to amplify the template sequence. LAMP uses a strand-displacing polymerase, and is isothermal, that is, it does not require heating and cooling cycles. The amplification process proceeds in an exponential manner (rather than a cyclic manner, like PCR) until all of the nucleotides (dATP, dTTP, dCTP dGTP, dUTP, and the like) in the reaction mixture have been incorporated into the amplified DNA. Optionally, an additional pair of primers may be included to accelerate the reaction. These primers, termed loop primers (LF and LB), hybridize to non-inner primer bound terminal loops of the inner primer dumbbell shaped products. Applications for LAMP have been further extended to include detection of RNA molecules by addition of reverse transcriptase (RT) enzyme. By including RNA detection, the types of targets for which LAMP can be applied are also expanded and add the ability to additionally target RNA based viruses, regulatory non-coding RNA (sRNA, miRNA), and RNA molecules that have been associated with particular disease or physiological states. The ability to detect RNA also has the potential to increase assay sensitivity, for instance, in choosing a highly expressed, stable, and/or abundant messenger RNA (mRNA) or ribosomal RNA (rRNA) targets. The preliminary phase of amplification involves the reverse transcription of RNA molecules to complementary DNA (cDNA). The cDNA then serves as template for the strand displacing DNA polymerase. Use of a thermostable RT enzyme enables the reaction to be completed at a single temperature and in a one step, single mix reaction.

In some embodiments, the method comprises primer set E-ID1. The primer set E-ID1 may comprise five primers having at least sequences of forward outer (F3), forward inner (FIP), loop forward (LF), backward outer (B3), and loop backward (LB).

In some embodiments, the method comprises primer set N-ID5, N-ID15, ID15n1L, S-ID17, S-ID24, or RdRp-ID37. The primer sets N-ID5, N-ID15, ID15n1L, S-ID17, S-ID24, and RdRp-ID37 may comprise six primers having at least sequences of forward outer (F3), forward inner (FIP), loop forward (LF), backward outer (B3), backward inner (BIP), and loop backward (LB).

RT-LAMP is a nucleic acid amplification method that relies on the addition of a reverse transcriptase, such as RTx Reverse Transcriptase or other reverse transcriptase enzymes, to the LAMP reaction to detect RNA target sequences. The amplified products are stem-loop structures with several repeated sequences of the target sequence and have multiple loops. The merit of this method is that denaturation of the DNA template is not required, and the LAMP and RT-LAMP reactions can occur under isothermal conditions (ranging from 35° C. to 75° C.).

LAMP is characterized by the use of four different primers specifically designed to recognize six distinct regions of the target gene. The four primers include two inner primers (forward inner primer (FIP) and backward inner primer (BIP)) and two out primers (forward outer primer (F3) and backward outer primer (B3)). A universal primer set may also include two additional primers, known as loop primers (loop forward (LF) and loop backward (LB)) primers. Although loop primers are optional, they may increase sensitivity and specificity of the amplification reaction. LAMP requires one enzyme and four to six primers that recognize six to eight distinct hybridization sites in the target sequence. The reaction can be accelerated with the addition of two additional primers, for a total of six primers. RT-LAMP requires two enzymes and four to six primers that recognize six to eight distinct hybridization sites in the target sequence. The method produces a large amount of amplified product, resulting in easier detection, such as detection by fluorescence or color of the reaction mixture. In some examples of the above embodiments, the RT-LAMP detects the target sequence in the sample and reverse transcribes the target sequence to a cDNA strand. In one embodiment, the amplification is detected involving a change in color or fluorescence visible to the naked eye. In some embodiments, the change of color is measured via a spectrophotometer or the fluorescence with a fluorescence spectrometer. As used herein, “fluorescence” refers to the emission of light by a substance that has absorbed light or other electromagnetic radiation and may include luminescence, photoluminescence, fluorescence, and phosphorescence.

In some examples, the target sequence is an RNA sequence of the SARS-CoV virus present in the sample. A “target sequence,” as used herein, refers to a nucleic acid sequence of the SARS-CoV genome, or a compliment thereof, that is amplified, detected, or both amplified and detected using one or more oligonucleotides herein provided. Additionally, while the term target sequence sometimes refers to a double stranded nucleic acid sequence, those skilled in the art will also recognize that the target sequence can also be single stranded, e.g., RNA, as is the case herein. In an embodiment, the target sequence may be a “target nucleic acid” which, as used herein, refers to any nucleic acid that is targeted by primers to determine its presence in a sample. A target sequence may be selected that is more or less specific to an entire genus, to more than one genus, to a species or subspecies, serogroup, auxotype, serotype, strain, isolate or other subset of organisms. In some embodiments, the sample includes a body fluid such as blood, saliva, sweat, urine, lymph, feces, etc., or a nasal swab, an oral swab obtained from a subject. In some examples, the subject is an animal. In some examples, the subject is a mammal. In one example, the mammal is a human. In some embodiments, the sample is a purified nucleic acid, a nucleic acid in a biological fluid, or a nucleic acid preparation in the swab or body fluid.

LAMP assays are described herein that enable rapid and sensitive detection of target nucleic acids, such as a nucleic acid of, or associated with, a pathogen, such as a virus, in a human or animal population. These assays are simple and portable while retaining sensitivity and minimizing false positives and negatives. The LAMP assays described herein rely on detection of a change in some aspect, such as fluorescence, or color of a dye in a reaction mix due to a change in pH or metal ion. Turbidity may also be used as an end point in some LAMP assays.

In some embodiments, the method further includes extracting nucleic acid from the sample. As used herein, the term “extracting” means obtaining, removing, or taking out with effort. In some embodiments, the extracting may include a separate protocol and a separate kit. The extracting may include disruption of a biological sample, including a tissue and/or cellular structure, denaturation of nucleoprotein complexes, inactivation of nucleases, and/or nucleic acid purification. In some embodiments, the extracting may be modified depending on the nucleic acid of interest and the sample source.

Detection of the RT-LAMP amplified products can be achieved via a variety of methods. In a preferred embodiment, the SARS assay is a colorimetric RT-LAMP assay and a fluorometric RT-LAMP assay. In some embodiments, the SARS assay is the colorimetric RT-LAMP assay. As used herein, the term “colorimetric test(s)” refers to detection of a target nucleic acid by a change in color of a dye in a reaction mix due to a change in pH or metal ion binding or dissociation. The assaying comprises mixing a master mix, a primer mix, the sample, and dH₂O to form a colorimetric RT-LAMP mixture; and incubating the colorimetric RT-LAMP mixture in a water bath at a constant temperature for at least 30 minutes. In some examples, the RT-LAMP mixture is incubated at a temperature between 40 to 70° C. until the color change is visible. In some embodiments, the color change is from pink to yellow and is visible by the natural eye. In some embodiments, the preferred temperature for the incubation is 65° C., and the preferred time for incubation is 30 minutes.

In some embodiments, the SARS assay is the fluorometric RT-LAMP assay. The assaying comprising mixing an RNA buffer, an aqueous salt solution, a dNTP solution, a fluorescent dye, an RNA enzyme, a primer mix, the sample, and dH₂O to form a fluorometric RT-LAMP mixture; and reacting the fluorometric RT-LAMP mixture in a PCR system at a constant temperature for a set number of cycles. In some embodiments, the set number of cycles may be 50 to 100 cycles, preferably 60 to 90 cycles, more preferably 70 to 80 cycles, and yet more preferably about 80 cycles. In some embodiments, the RT-LAMP amplified products are detected using intercalating dyes. Intercalating dyes are generally aromatic cations with planar structures that insert between stacked base pairs in the product duplex, an arrangement that provides an environmentally dependent fluorescence enhancement for dye molecules and creates a large increase in fluorescence signal relative to the dye free solution. In some embodiments, the SARS assay further comprises adding guanidine hydrochloride (GuHCl) to the colorimetric RT-LAMP assay and the fluorometric RT-LAMP assays. In some examples, the RNA buffer, the aqueous salt solution, the dNTP solution, the fluorescent dye, the RNA enzyme, and the primer mix are procured commercially, while in some examples, some or all the above components are prepared in a lab environment. In some embodiments, the SARS assay comprises adding guanidine hydrochloride, guanidine chloride, guanidine thiocyanate, guanidine sulfate, or a combination thereof to the colorimetric RT-LAMP assay and the fluorometric RT-LAMP assays. In some embodiments, adding GuHCl increases the detection sensitivity limit. In a preferred example, the set of oligonucleotide primers is E-ID1 (SEQ ID NO.: 37, SEQ ID NO.: 38, SEQ ID NO.: 39, SEQ ID NO.: 40, and SEQ ID NO.: 41), and the colorimetric RT-LAMP assay and the fluorometric RT-LAMP assay are made with the addition of guanidine hydrochloride (GuHCl). In some embodiments, the primer mix may include a nucleotide analog, for example, a phosphorothioate, an alkylphosphonothioate, or a peptide nucleic acid. In some examples, each primer may be modeled as a detection label to increase the detection. The detection label may be a compound, biomolecule, or biomolecule analog, etc., capable of confirming the density, concentration, amount, and the like, of an amplification product conventionally by linking, binding, or attaching to a primer, such as a biotin or a digoxigenin or alike can be added. In some examples, the specific length and sequence of the primer will depend on the conditions of use of the primer, such as temperature and ionic strength, as well as the complexity of the desired DNA or RNA target. In some embodiments, the mastermix may contain aptamers. The aptamers may be present for regulating the activity of transcriptase enzymes, RNAases, and the like.

The diagnostic LAMP methods described herein may be used for detecting pathogens, including any of: prokaryotes such as bacteria, eukaryotic pathogens such as multicellular parasites, fungi, single cell pathogen such as trypanosomes or yeasts, and Mycoplasma; as well as for use in genetic diseases, and in personalized medicine, which may require SNP detection or gene analysis of the genome of a subject or RNA analysis to determine gene expression profiles in response to an environmental or metabolic event. Other diagnostic uses include testing food for undesirable biological entities and monitoring environmental samples which may include biological material and are here referred to as biological samples also. The examples provided herein are directed to the SARS-CoV RNA virus; however, the method described herein may be applied to any colorimetric or fluorescent LAMP-based assay to detect a target nucleic acid and may find applications in non-LAMP assays as well.

The method of the present disclosure embodies the salient features of detecting the variants of the SARS-CoV virus. Thus, in an exemplary embodiment, the SARS-CoV is selected from the group consisting of SARS-CoV-2, SARS-CoV-HKU-39849 (e.g., AY278491.2), SARS-CoV-TW11 (e.g., AY502924.1), SARS-CoV-TW4 (e.g., AY502927.1), SARS-CoV-Sin846 (e.g., AY559094.1), SARS-CoV-GZ0402 (e.g., AY613947.1), and SARS-CoV-Tor2 (e.g., NC_004718.3). In one embodiment, the method and kit of the present disclosure can detect a mixture of SARS-CoV variants in the sample. In an embodiment, the method and kit of the present disclosure may detect any SARS-CoV variants, known and unknown in the art, in the sample.

In some embodiments, the SARS-CoV is SARS-CoV-2. The SARS-CoV-2 comprises at least one variant selected from the following group consisting of an alpha (B.1.1.7, Q.1-Q.8), beta (B.1.351, B.1.351.2, B.1.351.3), gamma (P.1, P.1.1, P.1.2), delta (B.1.617.2), and omicron (B.1.1.529). In some examples, the SARS-CoV-2 is detected among other viruses, including parainfluenza 3, enterovirus, rhinovirus, human metapneumovirus A+B, and parainfluenza virus 4, bocavirus, or coronavirus 229 E.

In some preferred embodiments, the method of the present disclosure includes amplifying isothermally the target sequence in the sample where the target sequence of the SARS-CoV nucleic acid sequence is located within at least one gene selected from the group consisting of nucleocapsid (N), spike(S), RNA-dependent RNA polymerase (RdRp), and envelope (E) of a SARS-CoV genome. In some embodiments, the set of oligonucleotide primers for the present method is selected from the group consisting of N-ID5 (Set-1), N-ID15 (Set-2), N-ID15n1L (Set-3), S-ID17 (Set-4), S-ID24 (Set-5), E-ID1 (Set-7), and RdRp-ID37 (Set-8). In an exemplary embodiment, the method includes the set of oligonucleotide primers selected from Set-1, Set-2, Set-3, Set-4, Set-5, and Set-8, each containing six oligonucleotide primers, the oligonucleotide primers having sequences of at least forward outer (F3), forward inner (FIP), loop forward (LF), backward outer (B3), backward inner (BIP), and loop backward (LB). In an example, the RT-LAMP reaction is initiated by annealing and extension of a pair of loop-forming primers (forward and backward inner primers, FIP and BIP, respectively), followed by annealing and extension of a pair of flanking primers (F3 and B3). Extension of these primers results in strand displacement of the loop-forming elements, which fold up to form terminal hairpin-loop structures. Once these structures have appeared, the amplification process becomes self-sustaining, and proceeds at a constant temperature in a continuous and exponential manner until the free nucleotides in the reaction mixture have been incorporated into the amplified sample. An additional pair of primers are included to accelerate the reaction. These primers, called loop primers (LF and LB), hybridize to non-inner primer bound terminal loops of the inner primer dumbbell shaped products. In a preferred example of the above embodiment, Set-7 contains five oligonucleotide primers, the oligonucleotide primers having sequences of at least forward outer (F3), forward inner (FIP), backward outer (B3), backward inner (BIP), and loop backward (LB), wherein the oligonucleotide primers have one or more nucleotide sequence that consist of SEQ ID NO.: 37, SEQ ID NO.: 38, SEQ ID NO.: 39, SEQ ID NO.: 40, and SEQ ID NO.: 41.

In some preferred embodiments, the method for detecting the presence of the amplification product for the target sequence of a SARS-CoV nucleic acid sequence includes no mis-amplifications of non-specific target regions leading to false-positive test results. In an embodiment, the set of oligonucleotide primers is Set-7, and the SARS assay has an accuracy of at least 94% for the colorimetric RT-LAMP assay and the fluorometric RT-LAMP assay, when compared to RT-qPCR assay results. In some embodiments, the RT-qPCR tests were performed with the kit described herein. In an embodiment, the kit may contain one or more primers targeting a human RP gene as a universal internal control. In another embodiment, the set of oligonucleotide primers is Set-7 and is used without mis-amplification and has a detection limit of 20 copies/μL. In yet another exemplary embodiment, the set of oligonucleotide primers is Set-7 and amplifies a conserved region in the SARS-CoV-2 E gene as early as 20 minutes with no cross-reactivity with other respiratory viruses, including other SARS.

In an exemplary embodiment of the present method, the set of oligonucleotide primers is Set-7. The sample's target sequence was amplified within 20 minutes and up to 120 minutes with no mis-amplification. In another example of the present method, the set of oligonucleotide primers is Set-7, and the SARS assay has a sensitivity of 89.5% for the colorimetric RT-LAMP assay and a sensitivity of 100% for the fluorometric RT-LAMP assay, when compared to RT-qPCR assay results. In yet another example of the present method, the set of oligonucleotide primers is Set-7, and the SARS assay has a specificity of at least 96% for the colorimetric RT-LAMP assay and the fluorometric RT-LAMP assay, when compared to RT-qPCR assay results.

The terms “sensitivity” and “specificity”, as used herein, have the meaning commonly understood in the art. Percent specificity is a measure of how well a test can identify true negatives in the sample. Specificity, or a true negative rate, is a measure of the probability of a negative test result, conditioned on the sample truly being negative. For example, if the biosensor of the present disclosure has a 100% specificity towards SARS-CoV virus, the biosensor would detect no false positive cases; i.e., samples without a target sequence of SARS-CoV would not be falsely identified as containing a target sequence of SARS-CoV. For further reference on specificity, see T. Fawcett, “An introduction to ROC analysis”, 2006, 27, 8, 861-874, incorporated herein by reference in its entirety.

In a preferred embodiment of the present disclosure, the method further comprises treating a subject for which the sample was obtained.

In one aspect of the present disclosure, a kit for detecting SARS-CoV in a sample is disclosed. Kits refer to a combination of materials that are needed to perform a reaction. The kit comprises a reverse transcriptase, a polymerase, a universal primer set suitable for loop-mediated isothermal amplification (LAMP) of the target sequence in a SARS-CoV nucleic acid sequence and variants thereof containing mutations within one or more primer binding sites. Further, the universal primer set suitable for LAMP is capable of hybridizing to the target sequence of a SARS-CoV nucleic acid sequence in the presence of a plurality of undefined mutations and is configured to provide a positive result for the target sequence of a SARS-CoV nucleic acid sequence in a predetermined assay period otherwise determined for a positive sample of a target nucleic acid having a known sequence. Further, the kit provides the reagents where any of the reagents in the kit may be combined in a mixture in a single container or provided in separate containers. In some embodiments, the kit may include a mixture of lyophilized reagents and reagents in a storage buffer. In one embodiment, the kit may contain multiple tubes with the master mix in a first tube, the primer mix in a second tube, and, optionally, other reagents in different tubes. In one example, the kit may analyze a plurality of different target nucleic acids. In addition, the kit may also include a printed and electronic user's guide describing the steps for performing the detection assay.

In a preferred embodiment, the method comprises Set-7 and the SARS assay shows zero mis-amplifications of non-template controls compared to a SARS assay with the primer sets of Set-1, Set-2, Set-3, Set-4, Set-5, and Set-8.

An advantage of the method of the present disclosure is that the method diagnoses SARS-CoV-2 within 20-30 minutes. Another advantage of the method is that the detection occurs without mis-amplification or false positive results eliminating any risk of misinterpreting the assay results. Another advantage of the present method is that the method is cheaper and faster, with the primer sets showing sensitivities of up to 90% and 100% on the colorimetric and fluorometric assays, respectively.

Accordingly, an aspect of the present disclosure is to provide primers and a method of detection of viruses of interest, including SARS-CoV-2. The kit of the present disclosure exhibited the advantage of the ready-to-use reagents for performing the detection of the viruses of interest, including SARS-CoV-2.

### Examples

The disclosure will now be illustrated with working examples intended to demonstrate the working of the disclosure and not to restrictively imply any limitations on the scope of the present disclosure. The working examples depict an example of the method of the present disclosure. Alignment of SARS-CoV-2 genome sequences and RT-LAMP primer design

SARS-CoV-2 whole genome sequences were downloaded from the GISAID database (Global Initiative on Sharing All Influenza Data, gisaid.org) sampled from different continents and included the globally dominant variants such as alpha (B.1.1.7, Q.1-Q.8), beta (B.1.351, B.1.351.2, B.1.351.3), gamma (P.1, P.1.1, P.1.2), delta (B.1.617.2), and omicron (B.1.1.529). In addition, the experiment included whole genome sequences of other SARS-CoV such as AY278491.2, AY502924.1, AY502927.1, AY559094.1, AY613947.1, and NC_004718.3 from the NCBI database, ncbi.nlm.nih.gov. A comparative analysis was made by aligning the multiple sequences using Clustal Omega, ebi.ac.uk. The mutation sites were identified using JalView (v2.11.1.3) program. All RT-LAMP primer sets were designed using the PrimerExplorer V5 program, primerexplorer.jp that target the conserved region in the N, S, RdRp, and E genes. In addition, the OligoAnalyzer tool from Integrated DNA Technologies (IDT), eu.idtdna.com, was used to check for unwanted secondary structures. The primers were synthesized by Alpha DNA (Montreal, Canada, alphadna.com). All the designed primer sets were tested on positive control (PC)—a mixture of verified high SARS-CoV-2-loaded specimens—and non-template control (NTC)—distilled water. In 25 μL reaction volume, 10× primer mix included 2 μM F3 and B3, 4 μM LF and LB, and 16 μM FIP and BIP.

### Colorimetric RT-LAMP

The colorimetric RT-LAMP mixture was composed of 12.5 μL WarmStart® Colorimetric LAMP 2× master mix with UDG (New England BioLabs), 2.5 μL 10× primer mix, 2 μL template (or dH₂O for NTC), and dH₂O up to 25 μL. The mixture was incubated in a water bath, Thermo Fisher Scientific (Waltham, MA, USA), set at 65° C. for 30 minutes or until a distinguishable color change was observed.

### Fluorometric RT-LAMP

The fluorometric RT-LAMP mixture contained 2.5 μL 10× LavaLAMP™ RNA buffer, 1.25 μL 100 mM MgSO₄, 2 μL 10 mM dNTP solution, 1 μL 20× Green Fluorescent Dye, 1 μL LavaLAMP™ RNA enzyme (Lucigen), 2.5 μL primer mix, 5 μL template (or dH₂O for NTC), and dH₂O up to 25 μL. Reactions were performed in a 7500 Fast Real-Time PCR System, Thermo Fisher Scientific (Waltham, MA, USA), at 70° C. for 80 cycles, 45-60 seconds using fluorescence activated molecule (FAM) filter.

### Agarose Gel Electrophoresis

RT-LAMP reactions were validated by loading reaction products in 2% agarose gel prepared with VisualaNA (A) DNA Stain from Molequle-On (Auckland, New Zealand) and run-in electrophoresis unit (Analytik Jena) for 45 minutes at 100 V. Then, the gel was visualized under a UV-transilluminator (ChemiDoc™ XRS+ System with Image Lab™ Software, Bio-Rad, USA). The amplifications in positive samples were visualized as ladder-type DNA bands.

### Collection of Samples and Validation of Results

In collaboration with the Microbiology Department of King Fahd University Hospital (KFUH), Dammam, which is authorized to store and analyze the SARS-CoV-2 samples from patients, 150 validated the SARS-CoV-2 positive or negative RNA samples were tested and validated with the herein developed a colorimetric RT-LAMP assay, a fluorometric RT-LAMP assay, and other commercially or in-house developed RT-qPCR kits (Tombuloglu, H., Sabit, H., Al-Khallaf, H., Kabanja, J. H., Alsaeed, M., Al-Saleh, N., & Al-Suhaimi, E. (2022). Multiplex real-time RT-PCR method for the diagnosis of SARS-CoV-2 by targeting viral N, RdRP and human RP genes. Scientific Reports, 12(1), 2853; and Tombuloglu, H., Sabit, H., Al-Suhaimi, E., Al Jindan, R., & Alkharsah, K. R. (2021). Development of multiplex real-time RT-PCR assay for the detection of SARS-CoV-2. PLOS ONE, 16(4), e0250942—each incorporated herein by reference in its entirety). The limit of detection (LoD) was determined using serial dilutions of a synthetic SARS-CoV-2 RNA control (Twist synthetic RNA control 51 (EPI_ISL_7718520), Twist Bioscience, USA). In addition, specificity was determined by testing other respiratory viruses' RNA extracted from clinical samples, including parainfluenza virus 3, enterovirus, rhinovirus, human metapneumovirus A+B, parainfluenza virus 4, bocavirus, and coronavirus 229 E.

### RT-PCR Assay

The positivity or negativity of the collected specimens was tested using the RT-qPCR to verify the RT-LAMP results by targeting two viral genes (RdRp, N, and E), a human RP gene as the internal control, and an NTC, as described earlier. The reactions were run in a real-time PCR instrument (Applied Biosystems™ real-time PCR 7500). The samples having a threshold cycle (Ct or Cq)≤37 with a sigmoidal amplification curve were positive; otherwise, they were considered negative.

### Statistical Analysis

The difference between the positive and negative color was determined spectrophotometrically by the mean values of the optical density (ΔOD) by transferring equal amounts of five positive and five negative clinical samples in a 96-well cell culture plate (Thermo Fisher Scientific) and reading absorbance values at 434 and 560 nm using a plate reader (BioTek Synergy HTX microplate reader, Agilent). The difference was considered statistically significant if the p-value <0.05 in an unpaired t-test performed using GraphPad Prism 9.0 (GraphPad Software, USA).

TABLE 1
Sequences of each RT-LAMP primer set used in this study.

| Primer | SEQ. | |
|---|---|---|
| set | ID No. | Sequence (5′ - 3′) |
| N-ID5 | 1 | F3: CCAGAATGGAGAACGCAGTG |
| (Set-1) | 2 | B3: CCGTCACCACCACGAATT |
| | 3 | FIP: AGCGGTGAACCAAGACGCAGGGCGCGATCAAAACAACG |
| | 4 | BIP: |
| | | AATTCCCTCGAGGACAAGGCGAGCTCTTCGGTAGTAGCCAA |
| | 5 | LF: TTATTGGGTAAACCTTGGGGC |
| | 6 | LB: TCCAATTAACACCAATAGCAGTCCA |
| N-ID15 | 7 | F3: AGATCACATTGGCACCCG |
| (Set-2) | 8 | B3: CCATTGCCAGCCATTCTAGC |
| | 9 | FIP: |
| | | TGCTCCCTTCTGCGTAGAAGCCAATGCTGCAATCGTGCTAC |
| | 10 | BIP: GGCGGCAGTCAAGCCTCTTCCCTACTGCTGCCTGGAGTT |
| | 11 | LF: GGCAATGTTGTTCCTTGAGGAAGTT |
| | 12 | LB: CACGTAGTCGCAACAGTTCAA |
| N- | 13 | F3: AGATCACATTGGCACCCG |
| ID15n1 | 14 | B3: CCATTGCCAGCCATTCTAGC |
| L | 15 | FIP: |
| (Set-3) | | TGCTCCCTTCTGCGTAGAAGCCAATGCTGCAATCGTGCTAC |
| | 16 | BIP: GGCGGCAGTCAAGCCTCTTCCCTACTGCTGCCTGGAGTT |
| | 17 | LF: GCAATGTTGTTCCTTGAGGAAGTT |
| | 18 | LB: GTTCCTCATCACGTAGTCGCAACA |
| S-ID17 | 19 | F3: TCTTTCACACGTGGTGTT |
| (Set-4) | 20 | B3: GTACCAAAAATCCAGCCTC |
| | 21 | FIP: |
| | | CATGGAACCAAGTAACATTGGAAAACCTGACAAAGTTTTCAG |
| | | ATCC |
| | 22 | BIP: |
| | | CTCTGGGACCAATGGTACTAAGAGGACTTCTCAGTGGAAGCA |
| | 23 | LF: GGTAAGAACAAGTCCTGAGTTGAA |
| | 24 | LB: GTTTGATAACCCTGTCCTACCATT |
| S-ID24 | 25 | F3: GGTGTTTATTACCCTGACAAAG |
| (Set-5) | 26 | B3: GTACCAAAAATCCAGCCTC |
| | 27 | FIP: CATGGAACCAAGTAACATTGGAAAATTTTCAGATCCTCAG |
| | | TTTTACATTC |
| | 28 | BIP: |
| | | CTCTGGGACCAATGGTACTAAGAGGACTTCTCAGTGGAAGCA |
| | 29 | LF: GAAAGGTAAGAACAAGTCCTGAGT |
| | 30 | LB: GTTTGATAACCCTGTCCTACCATT |
| S-set11 | 31 | F3: TCTATTGCCATACCCACAA |
| (Set-6) | 32 | B3: GTACCAAAAATCCAGCCTC |
| | 33 | FIP: |
| | | CATGGAACCAAGTAACATTGGAAAATTTTCAGATCCTCAGTTT |
| | | TACATTC |
| | 34 | BIP: |
| | | CTCTGGGACCAATGGTACTAAGAGGACTTCTCAGTGGAAGCA |
| | 35 | LF: GAAAGGTAAGAACAAGTCCTGAGT |
| | 36 | LB: GTTTGATAACCCTGTCCTACCATT |
| E-ID1 | 37 | F3: TCATTCGTTTCGGAAGAGA |
| (Set-7) | 38 | B3: AGGAACTCTAGAAGAATTCAGAT |
| | 39 | FIP: TGTAACTAGCAAGAATACCACGAAACAGGTACGTTAATAG |
| | | TTAATAGCG |
| | 40 | BIP: |
| | | GCTTCGATTGTGTGCGTACTCGAGAGTAAACGTAAAAAGAAG |
| | | G |
| | 41 | LB: GCTGCAATATTGTTAACGTGAGTC |
| RdRp- | 42 | F3: ACAAAGCCTTACATTAAGTGG |
| ID37 | 43 | B3: CACCATCAACAAATATTTTTCTCAC |
| (Set-8) | 44 | FIP: TGGGTGGTATGTCTGATCCCAATAGATTTGTTAAAATATGA |
| | | CTTCACGG |
| | 45 | BIP: |
| | | TGTGTTAACTGTTTGGATGACAGATTGTAAGTGGGAACACTGT |
| | 46 | LF: ACGGTCAAAGAGTTTTAACCTCTCT |
| | 47 | LB: GCATTCTGCATTGTGCAAACT |

F3: Forward outer, FIP: Forward inner, LF: Loop forward, B3: Backward outer, BIP: Backward inner, LB: Loop backward.

### RT-LAMP Primers' Performance

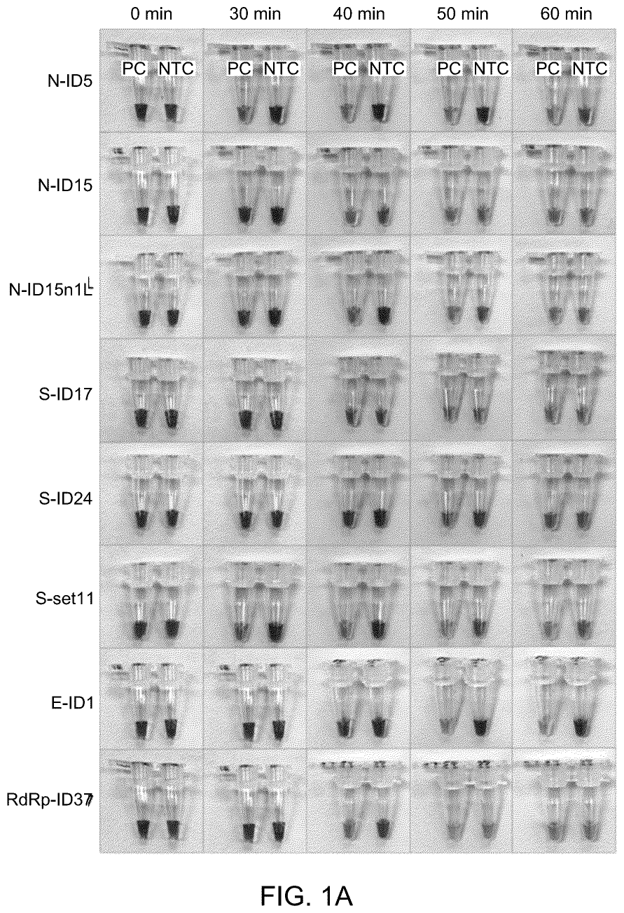

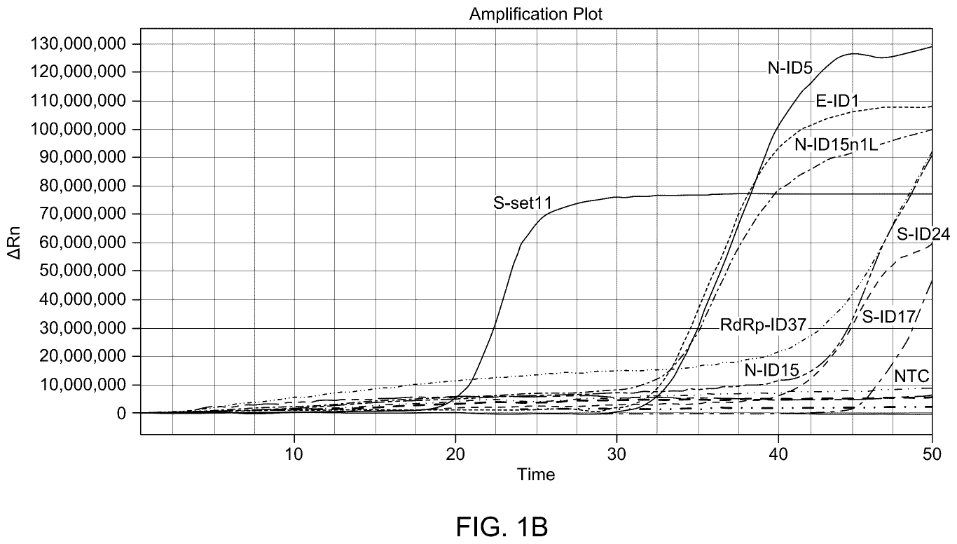

Five primer sets targeting N, E, S, or RdRp genes were tested on positive (PC) and non-template controls (NTC). In the colorimetric identification, color in all tubes was observed at 0 minutes (before reaction) and subsequently every ten minutes. According to the results shown in FIG. 1A, primer sets N-ID5 and S-set11 were the first to show a clear color difference after 30 minutes, followed by N-ID15n1L and RdRp-ID37 after 40 minutes. Primer set E-ID1 started to develop a color change at 50 min (without optimization), while most primer sets began to show mis-amplifications in NTC simultaneously. The reaction was terminated after 60 minutes due to developing false positivity in NTC in all the sets except E-ID1. Extending the time to over 120 minutes did not lead to mis-amplifications for the E-ID1 set harboring five primers. Therefore, this set was selected for further testing. The fluorometric results using the primer sets showing a fluorescent amplification in PC are depicted in FIG. 1B.

### Optimization of the Colorimetric and Fluorometric RT-LAMP Assays

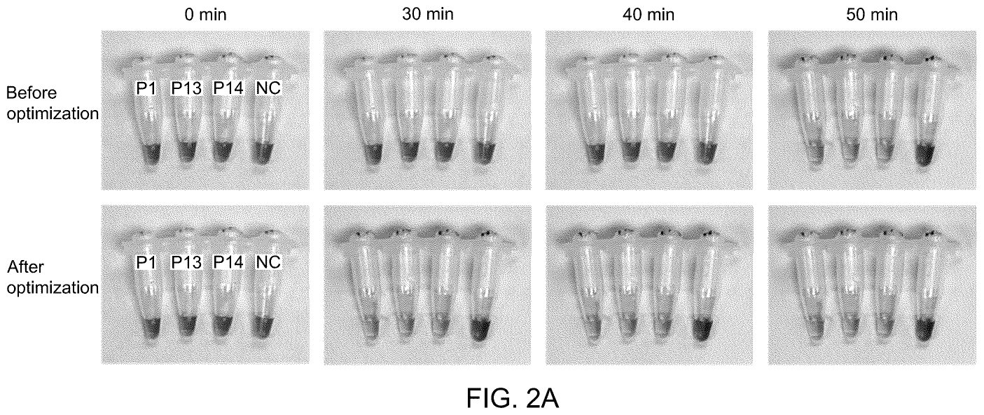

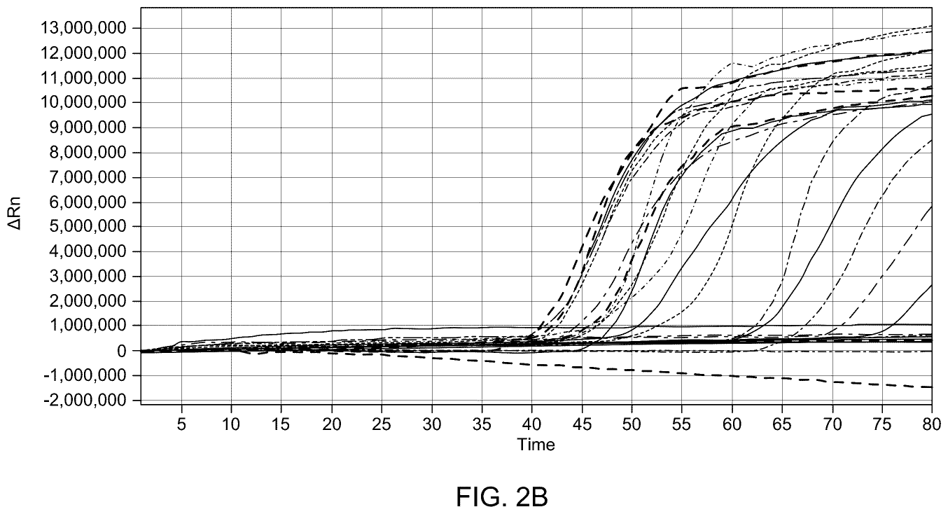

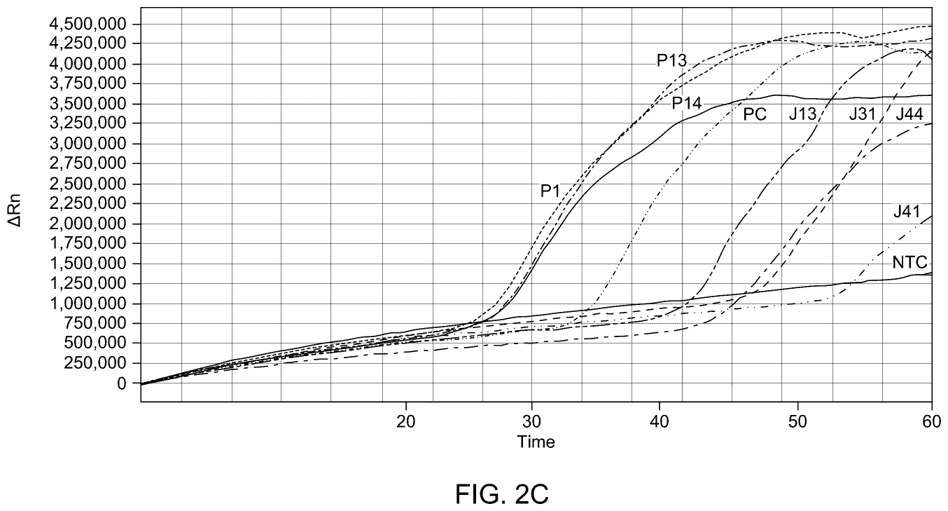

The colorimetric and fluorometric RT-LAMP assays were developed and optimized to enhance the performance of the E gene primers. The optimization includes the addition of GuHCl, testing different DNA polymerases (Bst 2.0 vs. Bst 3.0), and adjusting the optimum reaction temperature. In contrast to the detection time before optimization, the colorimetric assay showed an earlier color change within 30 minutes (FIG. 2A). Also, this improvement was seen in the fluorometric RT-LAMP assay, where it detected all positive samples in less than 60 minutes, an average of around 40 min (FIG. 2C), compared to its performance before optimization (FIG. 2B). The detection enhancement was found to be evident on comparing the FIG. 2B (before GuHCl) and FIG. 2C (after GuHCl).

### Limit-of-Detection (LoD) of E Gene Primers

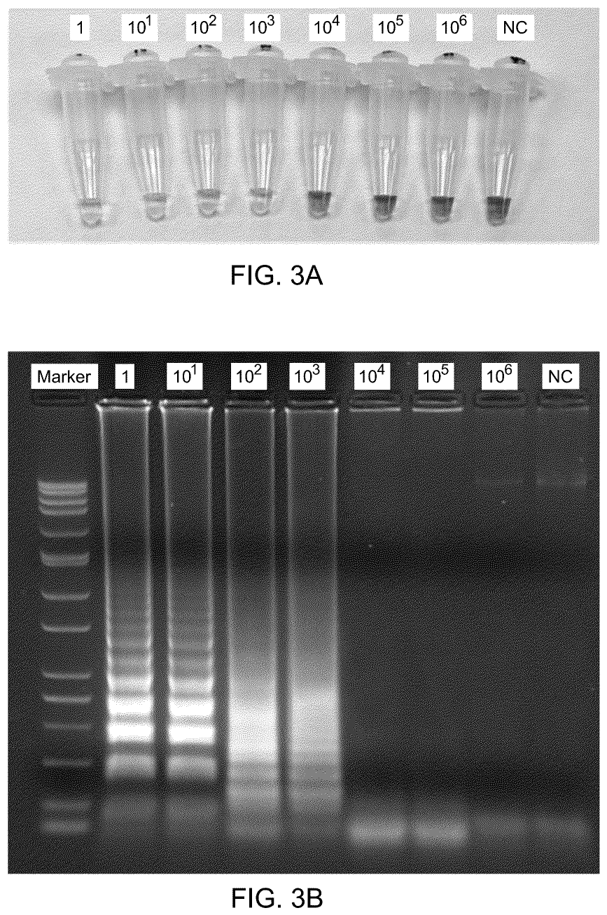

The SARS-CoV-2 synthetic RNA control (Twist synthetic RNA control 51 (EPI_ISL_7718520), Twist Bioscience, USA) was serially diluted (1, 10¹, 10², 10³, 10⁴, 10⁵, and 106 times) to determine the limit of detection (LoD) of RT-LAMP reactions. The color development to yellow is prominent in 1, 10¹, 10², and 10³-times dilutions, corresponding to 500 copies/reaction volume (25 μL) or 20 copies/μL for the 10³-times dilution (FIG. 3A). Each dilution was then loaded in 2% agarose and visualized under a UV trans-illuminator (FIG. 3B). The results indicate a ladder-type banding pattern in the detected dilutions. At the same time, no bands appeared in the others.

### RT-LAMP Assays on Clinical Samples

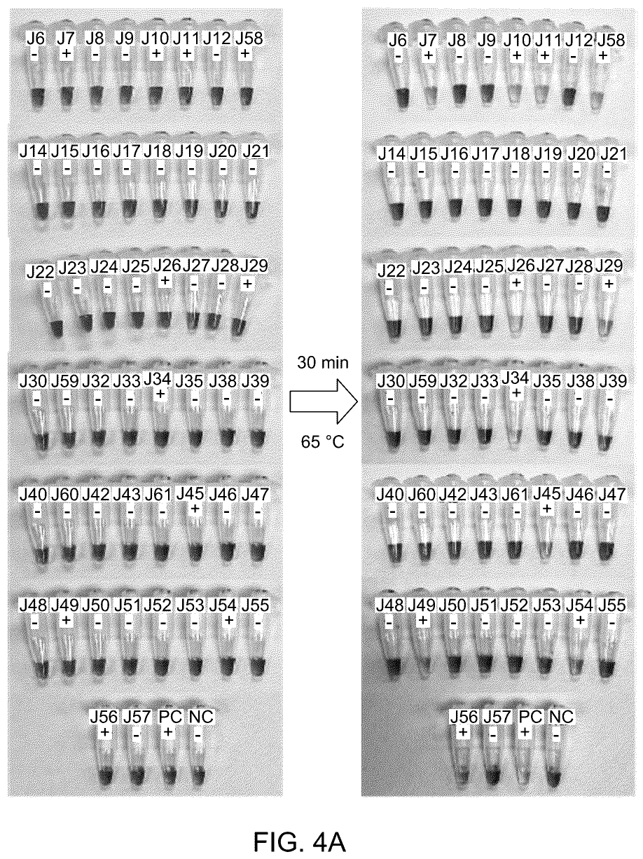

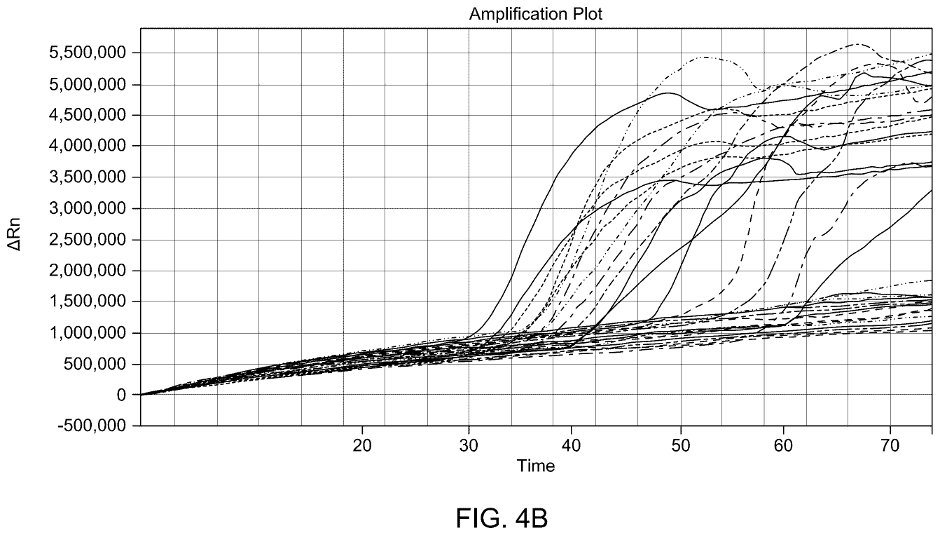

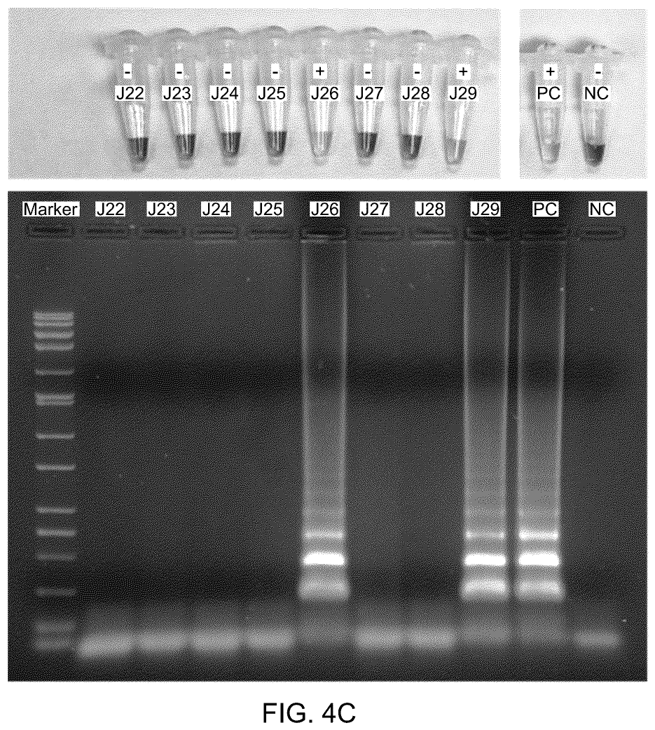

The performance of the primer set was tested on clinical specimens, as depicted in FIG. 4. In the colorimetric RT-LAMP, the tubes were incubated in a water bath at 65° C., and the positives started to turn yellow after 20 minutes (FIG. 4A). Eventually, there was 94.5% agreement between the colorimetric RT-LAMP and the RT-qPCR results. Similarly, clinical samples were fluorometrically tested using the optimized RT-LAMP protocol (FIG. 4B). The reaction was placed at 70° C. in a thermal cycler for 75 minutes. The fluorometric assay showed 98% agreement with the RT-qPCR results. All eight samples were loaded in 2% agarose stained with a DNA stain, and a 0.05-10 Kb DNA ladder was used as a marker, where positive samples showed DNA bands under a UV transilluminator (FIG. 4C).

### Sensitivity and Specificity of RT-LAMP Assays

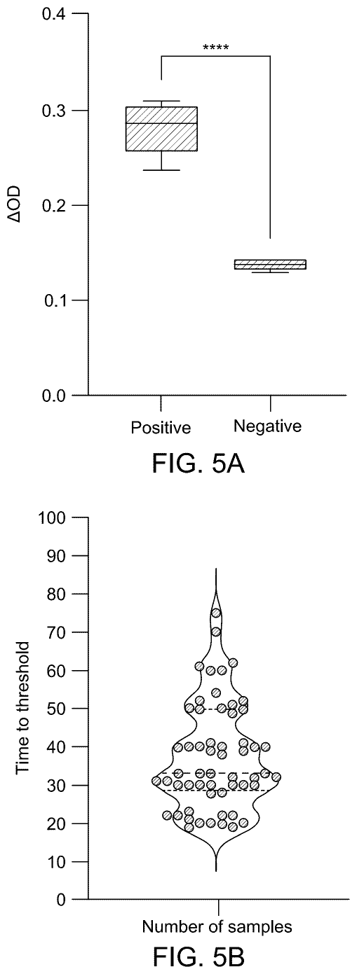

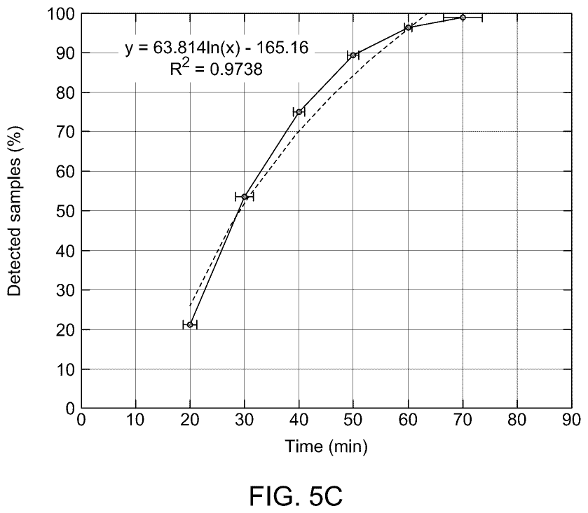

A total of 150 clinical specimens were tested in both assays. Accordingly, the colorimetric RT-LAMP assay had a sensitivity of 89.5%, specificity of 97.2%, and accuracy of 94.5%. Positive percent agreement (PPA) with the RT-qPCR was 94.4%, while negative percent agreement (NPA) was 94.6%. The fluorometric detection, the results revealed 100% sensitivity, 96.9% specificity, and 98% accuracy, with a PPA of 94.7% and an NPA of 100%. The color development in the positive and negative samples was quantified post-reaction spectrophotometrically by measuring the absorbance at 434 and 560 nm. The line inside the box represents the median, and the whiskers extend to the maximum and minimum values. The four asterisks (*) correspond to p<0.0001, which is a statistically significant difference (unpaired t-test, p<0.05, 95% CI). FIG. 5A shows that the difference in optical densities (ΔOD) between the positive yellow and negative red samples is statistically significant (p<0.0001, 95% CI). Furthermore, samples with high viral loads were detected in ˜20 minutes, while those with low viral loads took up to 75 minutes (FIG. 5B and FIG. 5C). On average, the detection time was calculated as 33 minutes, as indicated by dashed line in FIG. 5B. Besides, 90% of positives were detected within 50 minutes (FIG. 5C).

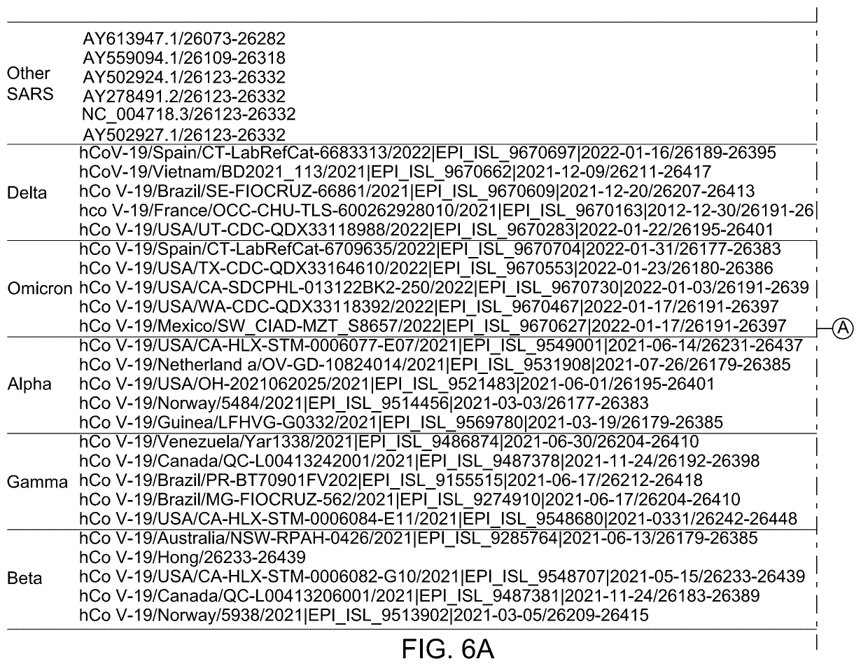

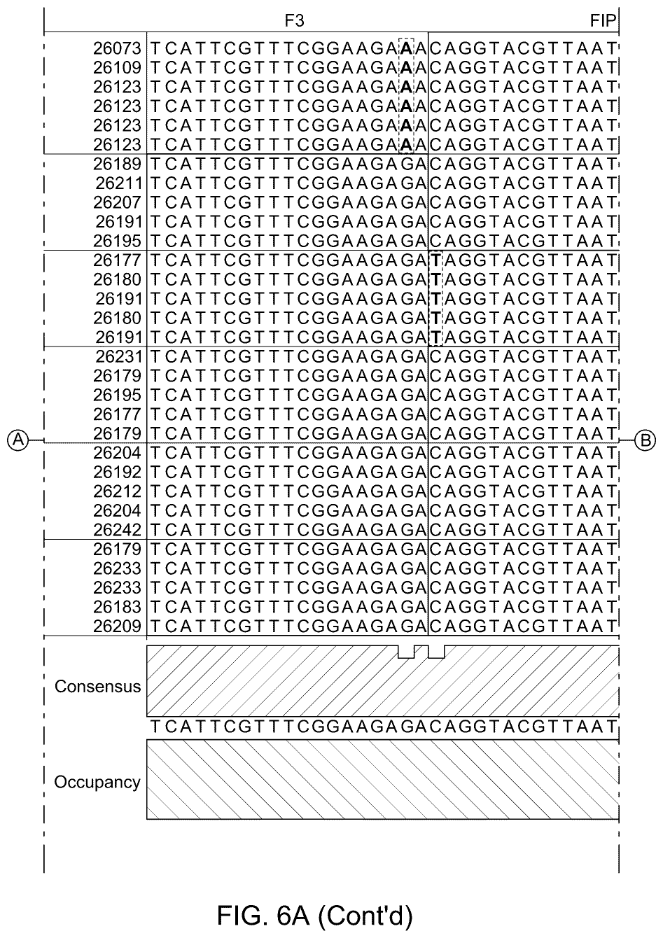

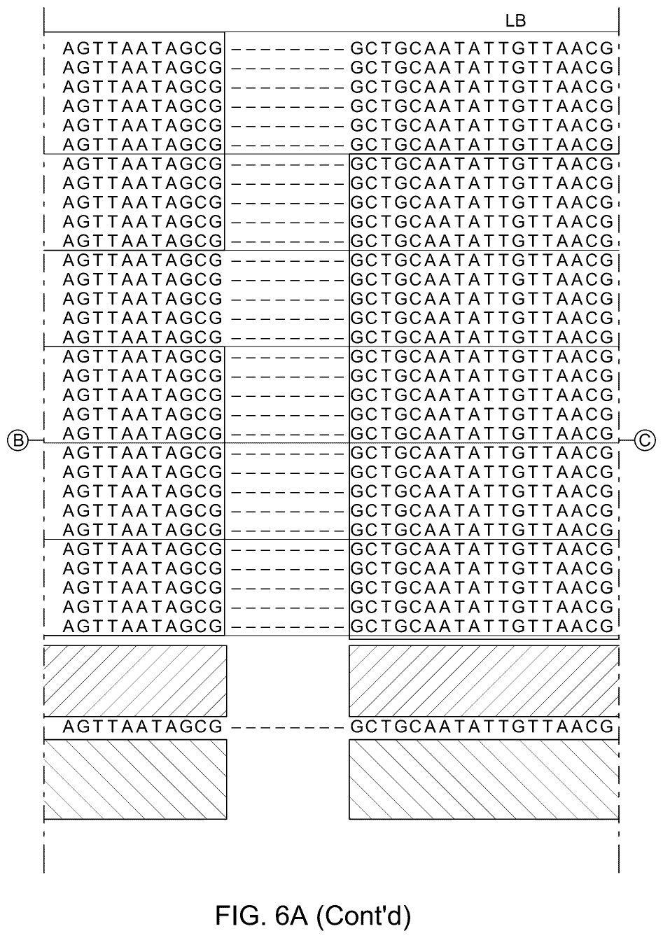

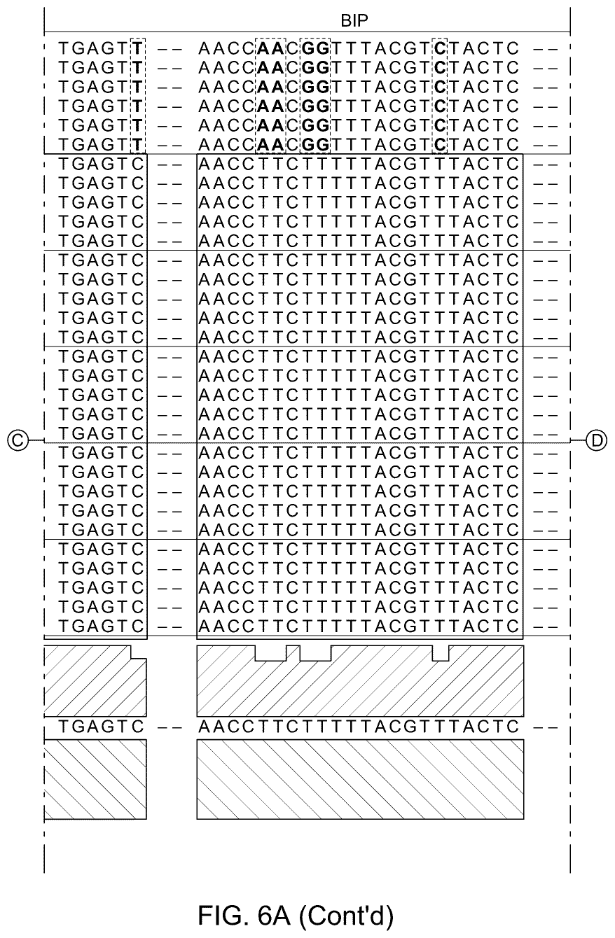

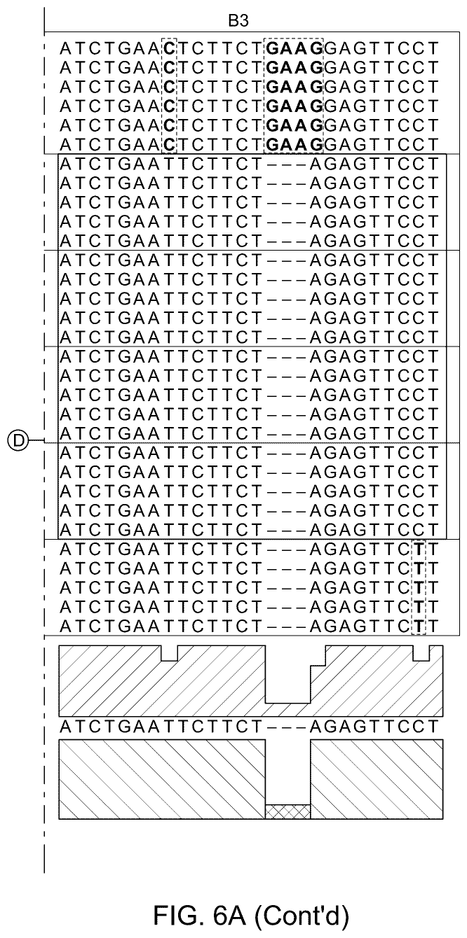

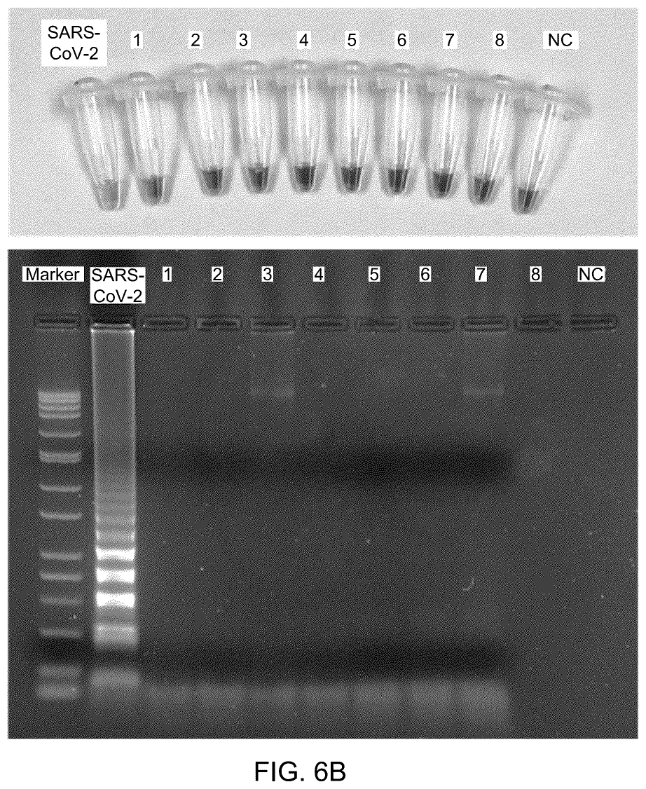

In addition, the inclusivity of the primers was tested in-silico by aligning the five VOC with other SARS viruses. The primers bind to the conserved regions in all the SARS-CoV-2 variants, including the recent omicron but are uncommon for the other SARS. These primer sequences bind to a conserved region in SARS-CoV-2 but are uncommon for other SARS. Only one point mutation exists in the forward inner primer (FIP) binding site (FIG. 6A). Also, the cross-reactivity was tested in-vitro against the most common respiratory viruses. The colorimetric assay only detected SARS-CoV-2 but none of the other respiratory viruses, which indicates high specificity against the diagnosis of COVID-19 infection (FIG. 6B). The other respiratory viruses include coronavirus 229 E, parainfluenza virus 3, parainfluenza virus 4, human metapneumovirus A+B, bocavirus, enterovirus, and rhinovirus.

Numerous modifications and variations of the present disclosure are possible in light of the above teachings. It is, therefore, to be understood that within the scope of the appended claims, the disclosure may be practiced otherwise than as specifically described herein.

1: A method of detecting SARS-CoV in a sample, comprising:
contacting the sample with a primer set and one or more reverse transcription polymerase chain reaction (RT-PCR) reagents to form a reaction mixture;
reverse transcribing a target sequence of a SARS-CoV nucleic acid sequence in the sample into complementary DNA (cDNA);
amplifying the cDNA by incubating the reaction mixture at a temperature and for a time sufficient to amplify the target sequence of the SARS-CoV nucleic acid sequence in the sample;
assaying the sample with a SARS assay to detect the amplified target sequence of the SARS-CoV nucleic acid sequence; and
detecting a presence of the amplified target sequence of the SARS-CoV nucleic acid sequence, thereby detecting SARS-CoV in the sample,
wherein the primer set is selected from the group consisting of:
N-ID5 (Set-1),
N-ID15 (Set-2),
N-ID15n1L (Set-3),
S-ID17 (Set-4),
S-ID24 (Set-5),
E-ID1 (Set-7), and
RdRp-ID37 (Set-8).

2: A method of detecting SARS-CoV in a sample, comprising:
contacting the sample with a primer set and one or more reverse transcription polymerase chain reaction (RT-PCR) reagents to form a reaction mixture;
reverse transcribing a target sequence of a SARS-CoV nucleic acid sequence in the sample into complementary DNA (cDNA);
amplifying the cDNA by incubating the reaction mixture at a temperature and for a time sufficient to amplify the target sequence of the SARS-CoV nucleic acid sequence in the sample;
assaying the sample with a SARS assay to detect the amplified target sequence of the SARS-CoV nucleic acid sequence; and
detecting a presence of the amplified target sequence of the SARS-CoV nucleic acid sequence, thereby detecting SARS-CoV in the sample,
wherein the primer set is S-set11 (Set-6).

3: The method of claim 1, wherein the primer set is E-ID1 and comprises five primers, the primers having at least sequences of forward outer (F3), forward inner (FIP), backward outer (B3), backward inner (BIP), and loop backward (LB).

4: The method of claim 1, wherein the primer set is N-ID5, N-ID15, ID15n1L, S-ID17, S-ID24, or RdRp-ID37 and comprises six primers, the primers having at least sequences of forward outer (F3), forward inner (FIP), loop forward (LF), backward outer (B3), backward inner (BIP), and loop backward (LB).

5: The method of claim 1, wherein the RT-PCR reagents comprise at least one reverse transcriptase, at least one deoxyribose nucleic acid (DNA) polymerase, deoxyribonucleotide triphosphates (dNTPs), magnesium ions, betaine or bovine serum albumin (BSA), a buffer, and optionally colorimetric and fluorometric dyes.

6: The method of claim 1, wherein the amplifying is a reverse-transcription loop-mediated isothermal amplification (RT-LAMP) process.

7: The method of claim 1, further comprising:
extracting nucleic acid from the sample.

8: The method of claim 1, wherein the SARS assay is a colorimetric RT-LAMP assay and a fluorometric RT-LAMP assay.

9: The method of claim 8, wherein the SARS assay is the colorimetric RT-LAMP assay, wherein the assaying comprises:
mixing a master mix, a primer mix, the sample, and dH₂O to form a colorimetric RT-LAMP mixture; and
incubating the colorimetric RT-LAMP mixture in a water bath at a constant temperature for at least 30 minutes.

10: The method of claim 8, wherein the SARS assay is the fluorometric RT-LAMP assay, wherein the assaying comprises:
mixing an RNA buffer, an aqueous salt solution, a dNTP solution, a fluorescent dye, an RNA enzyme, a primer mix, the sample, and dH₂O to form a fluorometric RT-LAMP mixture; and
reacting the fluorometric RT-LAMP mixture in a PCR system at a constant temperature for a set number of cycles.

11: The method of claim 8, wherein the SARS assay further comprises adding guanidine hydrochloride (GuHCl) to the colorimetric RT-LAMP assay and the fluorometric RT-LAMP assay.

12: The method of claim 1, wherein the SARS-CoV is selected from the group consisting of SARS-CoV-2, SARS-CoV-HKU-39849, SARS-CoV-TW11, SARS-CoV-TW4, SARS-CoV-Sin846, SARS-CoV-GZ0402, and SARS-CoV-Tor2.

13: The method of claim 1, wherein the SARS-CoV is SARS-CoV-2, and wherein the SARS-CoV-2 comprises at least one variant selected from the following group consisting of alpha (B.1.1.7, Q.1-Q.8), beta (B.1.351, B.1.351.2, B.1.351.3), gamma (P.1, P.1.1, P.1.2), delta (B.1.617.2), and Omicron (B.1.1.529).

14: The method of claim 1, wherein the target sequence of the SARS-CoV nucleic acid sequence is located within at least one gene selected from the group consisting of nucleocapsid (N), spike(S), RNA-dependent RNA polymerase (RdRp), and envelope (E) of a SARS-CoV genome.

15: The method of claim 1, wherein the detection of the presence of the amplification product for the target sequence of a SARS-CoV nucleic acid sequence includes an accuracy of at least 94% for the colorimetric RT-LAMP assay and the fluorometric RT-LAMP assay, wherein accuracy is based on colorimetric RT-LAMP assay results and fluorometric RT-LAMP assay results compared to reverse transcription real-time polymerase chain reaction (RT-qPCR) assay results.

16: The method of claim 1, wherein the primer set is E-ID1 and has a limit of detection of 20 copies/μL.

17: The method of claim 1, wherein the primer set is E-ID1 and amplifies a conserved region in the SARS-CoV-2 E gene as early as 20 minutes with no cross-reactivity with other respiratory viruses, including other SARS, wherein the SARS assay has a specificity of at least 96% for the colorimetric RT-LAMP assay and the fluorometric RT-LAMP assay, wherein specificity is based on colorimetric RT-LAMP assay results and fluorometric RT-LAMP assay results compared to RT-qPCR assay results.

18: The method of claim 1, wherein the primer set is E-ID1 and the target sequence in the sample was amplified within 20 minutes and up to 120 minutes, wherein the SARS assay has a sensitivity of at least 89% for the colorimetric RT-LAMP assay and the fluorometric RT-LAMP assay, wherein sensitivity is based on colorimetric RT-LAMP assay results and fluorometric RT-LAMP assay results compared to RT-qPCR assay results.

19: The method of claim 1, wherein the method further comprises treating a subject for which the sample was obtained.

20: A kit for detection of SARS-CoV in a sample, the kit comprising:
a reverse transcriptase, a polymerase, a universal primer set for loop-mediated isothermal amplification (LAMP) of the target sequence in a SARS-CoV nucleic acid sequence and variants thereof containing mutations within one or more primer binding sites;
wherein the universal primer set suitable for LAMP hybridizes to the target sequence of a SARS-CoV nucleic acid sequence in the presence of a plurality of undefined mutations, and is configured to provide a positive result for the target sequence of a SARS-CoV nucleic acid sequence in a predetermined assay time period otherwise determined for a positive sample of a target nucleic acid having a known sequence;
wherein any of the reagents in the kit may be combined in a mixture in a single container or provided in separate containers.


SEQUENCE LISTING (55 sequences)

SEQ ID NO: 1 - RNA, 20 nt, synthetic construct
  source 1..20: mol_type = other RNA
ccagaatggagaacgcagtg

SEQ ID NO: 2 - RNA, 18 nt, synthetic construct
  source 1..18: mol_type = other RNA
ccgtcaccaccacgaatt

SEQ ID NO: 3 - RNA, 38 nt, synthetic construct
  source 1..38: mol_type = other RNA
agcggtgaaccaagacgcagggcgcgatcaaaacaacg

SEQ ID NO: 4 - RNA, 41 nt, synthetic construct
  source 1..41: mol_type = other RNA
aattccctcgaggacaaggcgagctcttcggtagtagccaa

SEQ ID NO: 5 - RNA, 21 nt, synthetic construct
  source 1..21: mol_type = other RNA
ttattgggtaaaccttggggc

SEQ ID NO: 6 - RNA, 25 nt, synthetic construct
  source 1..25: mol_type = other RNA
tccaattaacaccaatagcagtcca

SEQ ID NO: 7 - RNA, 18 nt, synthetic construct
  source 1..18: mol_type = other RNA
agatcacattggcacccg

SEQ ID NO: 8 - RNA, 20 nt, synthetic construct
  source 1..20: mol_type = other RNA
ccattgccagccattctagc

SEQ ID NO: 9 - RNA, 41 nt, synthetic construct
  source 1..41: mol_type = other RNA
tgctcccttctgcgtagaagccaatgctgcaatcgtgctac

SEQ ID NO: 10 - RNA, 39 nt, synthetic construct
  source 1..39: mol_type = other RNA
ggcggcagtcaagcctcttccctactgctgcctggagtt

SEQ ID NO: 11 - RNA, 25 nt, synthetic construct
  source 1..25: mol_type = other RNA
ggcaatgttgttccttgaggaagtt

SEQ ID NO: 12 - RNA, 21 nt, synthetic construct
  source 1..21: mol_type = other RNA
cacgtagtcgcaacagttcaa

SEQ ID NO: 13 - RNA, 18 nt, synthetic construct
  source 1..18: mol_type = other RNA
agatcacattggcacccg

SEQ ID NO: 14 - RNA, 20 nt, synthetic construct
  source 1..20: mol_type = other RNA
ccattgccagccattctagc

SEQ ID NO: 15 - RNA, 41 nt, synthetic construct; identical to SEQ ID NO: 9
  source 1..41: mol_type = other RNA

SEQ ID NO: 16 - RNA, 39 nt, synthetic construct; identical to SEQ ID NO: 10
  source 1..39: mol_type = other RNA

SEQ ID NO: 17 - RNA, 24 nt, synthetic construct
  source 1..24: mol_type = other RNA
gcaatgttgttccttgaggaagtt

SEQ ID NO: 18 - RNA, 24 nt, synthetic construct
  source 1..24: mol_type = other RNA
gttcctcatcacgtagtcgcaaca

SEQ ID NO: 19 - RNA, 18 nt, synthetic construct
  source 1..18: mol_type = other RNA
tctttcacacgtggtgtt

SEQ ID NO: 20 - RNA, 19 nt, synthetic construct
  source 1..19: mol_type = other RNA
gtaccaaaaatccagcctc

SEQ ID NO: 21 - RNA, 46 nt, synthetic construct
  source 1..46: mol_type = other RNA
catggaaccaagtaacattggaaaacctgacaaagttttcagatcc

SEQ ID NO: 22 - RNA, 42 nt, synthetic construct
  source 1..42: mol_type = other RNA
ctctgggaccaatggtactaagaggacttctcagtggaagca

SEQ ID NO: 23 - RNA, 24 nt, synthetic construct
  source 1..24: mol_type = other RNA
ggtaagaacaagtcctgagttgaa

SEQ ID NO: 24 - RNA, 24 nt, synthetic construct
  source 1..24: mol_type = other RNA
gtttgataaccctgtcctaccatt

SEQ ID NO: 25 - RNA, 22 nt, synthetic construct
  source 1..22: mol_type = other RNA
ggtgtttattaccctgacaaag

SEQ ID NO: 26 - RNA, 19 nt, synthetic construct
  source 1..19: mol_type = other RNA
gtaccaaaaatccagcctc

SEQ ID NO: 27 - RNA, 50 nt, synthetic construct
  source 1..50: mol_type = other RNA
catggaaccaagtaacattggaaaattttcagatcctcagttttacattc

SEQ ID NO: 28 - RNA, 42 nt, synthetic construct; identical to SEQ ID NO: 22
  source 1..42: mol_type = other RNA

SEQ ID NO: 29 - RNA, 24 nt, synthetic construct
  source 1..24: mol_type = other RNA
gaaaggtaagaacaagtcctgagt

SEQ ID NO: 30 - RNA, 24 nt, synthetic construct
  source 1..24: mol_type = other RNA
gtttgataaccctgtcctaccatt

SEQ ID NO: 31 - RNA, 19 nt, synthetic construct
  source 1..19: mol_type = other RNA
tctattgccatacccacaa

SEQ ID NO: 32 - RNA, 23 nt, synthetic construct
  source 1..23: mol_type = other RNA
ggtgttttgtaaatttgtttgac

SEQ ID NO: 33 - RNA, 47 nt, synthetic construct
  source 1..47: mol_type = other RNA
cattcagttgaatcaccacaaatgtgtgttaccacagaaattctacc

SEQ ID NO: 34 - RNA, 45 nt, synthetic construct
  source 1..45: mol_type = other RNA
gttgcaatatggcagtttttgtacattgggtgtttttgtcttgtt

SEQ ID NO: 35 - RNA, 24 nt, synthetic construct
  source 1..24: mol_type = other RNA
actgatgtcttggtcatagacact

SEQ ID NO: 36 - RNA, 25 nt, synthetic construct
  source 1..25: mol_type = other RNA
taaaccgtgctttaactggaatagc

SEQ ID NO: 37 - RNA, 19 nt, synthetic construct
  source 1..19: mol_type = other RNA
tcattcgtttcggaagaga

SEQ ID NO: 38 - RNA, 23 nt, synthetic construct
  source 1..23: mol_type = other RNA
aggaactctagaagaattcagat

SEQ ID NO: 39 - RNA, 49 nt, synthetic construct
  source 1..49: mol_type = other RNA
tgtaactagcaagaataccacgaaacaggtacgttaatagttaatagcg

SEQ ID NO: 40 - RNA, 43 nt, synthetic construct
  source 1..43: mol_type = other RNA
gcttcgattgtgtgcgtactcgagagtaaacgtaaaaagaagg

SEQ ID NO: 41 - RNA, 24 nt, synthetic construct
  source 1..24: mol_type = other RNA
gctgcaatattgttaacgtgagtc

SEQ ID NO: 42 - RNA, 21 nt, synthetic construct
  source 1..21: mol_type = other RNA
acaaagccttacattaagtgg

SEQ ID NO: 43 - RNA, 25 nt, synthetic construct
  source 1..25: mol_type = other RNA
caccatcaacaaatatttttctcac

SEQ ID NO: 44 - RNA, 49 nt, synthetic construct
  source 1..49: mol_type = other RNA
tgggtggtatgtctgatcccaatagatttgttaaaatatgacttcacgg

SEQ ID NO: 45 - RNA, 43 nt, synthetic construct
  source 1..43: mol_type = other RNA
tgtgttaactgtttggatgacagattgtaagtgggaacactgt

SEQ ID NO: 46 - RNA, 25 nt, synthetic construct
  source 1..25: mol_type = other RNA
acggtcaaagagttttaacctctct

SEQ ID NO: 47 - RNA, 21 nt, synthetic construct
  source 1..21: mol_type = other RNA
gcattctgcattgtgcaaact

SEQ ID NO: 48 - RNA, 19 nt, synthetic construct
  source 1..19: mol_type = other RNA
tcattcgtttcggaagaaa

SEQ ID NO: 49 - RNA, 24 nt, synthetic construct
  source 1..24: mol_type = other RNA
taggtacgttaatagttaatagcg

SEQ ID NO: 50 - RNA, 24 nt, synthetic construct
  source 1..24: mol_type = other RNA
gctgcaatattgttaacgtgagtt

SEQ ID NO: 51 - RNA, 22 nt, synthetic construct
  source 1..22: mol_type = other RNA
aaccttctttttacgtttactc

SEQ ID NO: 52 - RNA, 22 nt, synthetic construct
  source 1..22: mol_type = other RNA
aaccaacggtttacgtctactc

SEQ ID NO: 53 - RNA, 23 nt, synthetic construct
  source 1..23: mol_type = other RNA
atctgaattcttctagagttcct

SEQ ID NO: 54 - RNA, 26 nt, synthetic construct
  source 1..26: mol_type = other RNA
atctgaactcttctgaaggagttcct

SEQ ID NO: 55 - RNA, 23 nt, synthetic construct
  source 1..23: mol_type = other RNA
atctgaattcttctagagttctt